(19) United States
(12) Patent Application Publication
(10) Pub. No.: US 2008/0103340 A1
(43) Pub. Date: May 1, 2008

(54) APPLICATIONS OF BIOBASED GLYCOL COMPOSITIONS

(75) Inventors: Thomas Paul Binder, Decatur, IL (US); Paul D. Bloom, Decatur, IL (US); George B. Poppe, Forsyth, IL (US)

Correspondence Address: KIRKPATRICK & LOCKHART PRESTON GATES ELLIS LLP;HENRY W. OLIVER BUILDING, 535 SMITHFIELD STREET, PITTSBURGH, PA, 15222, US

(73) Assignee: Archer-Daniels-Midland Company, Decatur, IL (US)

(21) Appl. No.: 11/614,349

(22) Filed: Dec. 21, 2006

Related U.S. Application Data
(60) Provisional application No. 60/854,949, filed on Oct. 27, 2006.

(51) Int. Cl. C07C 31/18

(52) U.S. Cl. 568/863

(57) ABSTRACT

A biobased replacement for propylene glycol and ethylene glycol derived from petrochemical sources is presented. The product mixture from the hydrogenolysis of certain polyols from biobased renewable resources may replace propylene glycol and ethylene glycol products from petrochemical sources. Applications and methods of the biobased hydrogenolysis product mixture are disclosed. The compositions and methods provide a feedstock for industrial use which has a ¹³C/¹²C isotope ratio characteristic of bioderived material.

### CROSS REFERENCE TO RELATED APPLICATIONS

This application claims the benefit of U.S. Provisional Application No., 60/854,949, filed Oct. 27, 2006, the disclosure of which is incorporated by this reference.

### TECHNICAL FIELD

The present disclosure provides a biobased replacement for propylene glycol and ethylene glycol derived from petrochemical sources comprising a biobased hydrogenolysis product mixture. Applications and methods of the biobased hydrogenolysis product mixture are disclosed.

### BACKGROUND

Propylene glycol is an organic polyol compound having the structure designated by the IUPAC name 1,2-dihydroxypropane. Ethylene glycol is an organic polyol having the structure designated by the IUPAC name 1,2-dihydroxyethane. Propylene glycol and ethylene glycol are used as feedstocks or raw materials for many industrial processes. Millions of pounds of propylene glycol and ethylene glycol are produced and used every year.

Typically, propylene glycol and ethylene glycol are produced from petrochemical sources. For example, commercial production of propylene glycol may involve the hydration of propylene oxide, which is made by the oxidation of propylene. Similarly, commercial production of ethylene glycol may involve the hydration of ethylene oxide, made by the oxidation of ethylene. Both propylene and ethylene are industrial by-products of gasoline manufacture, for example as by-products of fluid cracking of gas oils or steam cracking of hydrocarbons.

The world's supply of petroleum is being depleted at an increasing rate. Eventually, demand for petrochemical derived products will outstrip the supply of available petroleum. When this occurs, the market price of petroleum and, consequently, petroleum derived products will likely increase, making products derived from petroleum more expensive and less desirable. As the available supply of petroleum decreases, alternative sources and, in particular, renewable sources of comparable products will necessarily have to be developed. One potential renewable source of petroleum derived products is products derived from biobased matter, such as agricultural and forestry products Use of biobased products may potentially counteract, at least in part, the problems associated with depletion of the petroleum supply.

In an effort to diminish dependence on petroleum products the United States government enacted the Farm Security and Rural Investment Act of 2002, section 9002 (7 U.S.C. 8102), hereinafter “FRISA”, which requires federal agencies to purchase biobased products, if available, for all items costing over $10,000 In response, the United States Department of Agriculture (“USDA”) has developed Guidelines for Designating Biobased Products for Federal Procurement (7 C.F.R. §2902) to implement FRISA, including the labeling of biobased products with a “U.S.D.A. Certified Biobased Product” label.

As used herein, the term “bioderived” means derived from or synthesized by a renewable biological feedstock, such as, for example, an agricultural, forestry, plant, bacterial, or animal feedstock. As used herein, the term “biobased” means a product that is composed, in whole or in significant part, of biological products or renewable agricultural materials (including plant, animal and marine materials) or forestry materials. As used herein, the term “petroleum derived” means a product derived from or synthesized from petroleum or a petrochemical feedstock.

FRISA has established certification requirements for determining biobased content. These methods require the measurement of variations in isotopic abundance between biobased products and petroleum derived products, for example, by liquid scintillation counting, accelerator mass spectrometry, or high precision isotope ratio mass spectrometry. Isotopic ratios of the isotopes of carbon, such as the ¹³C/¹²C carbon isotopic ratio or the ¹⁴C/¹²C carbon isotopic ratio, can be determined using analytical methods, such as isotope ratio mass spectrometry, with a high degree of precision. Studies have shown that isotopic fractionation due to physiological processes, such as, for example, CO₂ transport within plants during photosynthesis, leads to specific isotopic ratios in natural or bioderived compounds. Petroleum and petroleum derived products have a different ¹³C/¹²C carbon isotopic ratio due to different chemical processes and isotopic fractionation during the generation of petroleum. In addition, radioactive decay of the unstable ¹⁴C carbon radioisotope leads to different isotope ratios in biobased products compared to petroleum products. Biobased content of a product may be verified by ASTM International Radioisotope Standard Method D 6866. ASTM International Radioisotope Standard Method D 6866 determines biobased content of a material based on the amount of biobased carbon in the material or product as a percent of the weight (mass) of the total organic carbon in the material or product. Both bioderived and biobased products will have a carbon isotope ratio characteristic of a biologically derived composition.

Biology offers an attractive alternative for industrial manufacturers looking to reduce or replace their reliance on petrochemicals and petroleum derived products. The replacement of petrochemicals and petroleum derived products with products and/or feedstocks derived from biological sources (i.e., biobased products) offer many advantages. For example, products and feedstocks from biological sources are typically a renewable resource. As the supply of easily extracted petrochemicals continue to be depleted, the economics of petrochemical production will likely force the cost of the petrochemicals and petroleum derived products to higher prices compared to biobased products. In addition, companies may benefit from the marketing advantages associated with bioderived products from renewable resources in the view of a public becoming more concerned with the supply of petrochemicals.

### SUMMARY

The various embodiments of the present disclosure provide biobased compositions for the replacement of petroleum derived propylene glycol and/or ethylene glycol in various applications.

According to one embodiment, the present disclosure provides a composition for use as a replacement for petroleum derived propylene glycol or ethylene glycol. The composition comprises a hydrogenolysis product of a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof. The hydrogenolysis product comprises a mixture of propylene glycol, ethylene glycol, and one or more of methanol, 2-propanol, glycerol, lactic acid, glyceric acid, butanediols, sodium lactate, and sodium glycerate. The composition is 100% biobased as determined by ASTM International Radioisotope Standard Method D 6866.

Other embodiments provide methods of making a bioderived composition for use as a replacement for petroleum derived propylene glycol or ethylene glycol. The methods comprise reacting a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof, via a hydrogenolysis process to give a hydrogenolysis product comprising a mixture of propylene glycol, ethylene glycol, and one or more of methanol, 2-propanol, glycerol, lactic acid, glyceric acid, butanediols, sodium lactate, and sodium glycerate; and adding the hydrogenolysis product to a formulation as a replacement for petroleum derived propylene glycol or petroleum derived ethylene glycol. The hydrogenolysis product is 100% biobased as determined by ASTM International Radioisotope Standard Method D 6866.

Still other embodiments provide methods for making a bioderived polyester polymer. The methods comprise mixing a hydrogenolysis product with one of a bioderived saturated dicarboxylic acid monomer reagent and a bioderived unsaturated dicarboxylic acid monomer reagent to form a reaction mixture; and reacting the reaction mixture to afford the bioderived polyester polymer. The hydrogenolysis product is produced by hydrogenolysis of a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof, and comprises a mixture of propylene glycol, ethylene glycol, and one or more of methanol, 2-propanol, glycerol, lactic acid, glyceric acid, butanediols, sodium lactate, and sodium glycerate. The bioderived polyester polymer is from 50% to 100% biobased as determined by ASTM International Radioisotope Standard Method D 6866.

Further embodiments provide methods for making a bioderived ester. The methods comprise reacting a hydrogenolysis product with one of a fatty acid methyl ester, a carboxylic acid and a triglyceride. The hydrogenolysis product is produced by hydrogenolysis of a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof, and comprises a mixture of propylene glycol, ethylene glycol, and one or more of methanol, 2-propanol, glycerol, lactic acid, glyceric acid, butanediols, sodium lactate, and sodium glycerate. The bioderived ester is 100% biobased as determined by ASTM International Radioisotope Standard Method D 6866.

### BRIEF DESCRIPTION OF DRAWINGS

The various embodiments of the present disclosure will be better understood when read in conjunction with the following figures.

FIG. 1 illustrates certain approaches to modifying the unsaturated polyester polymers of the present disclosure.

FIG. 2 illustrates one embodiment for the synthesis of the mixed polyol lactate esters of the present disclosure.

FIG.3 illustrates one embodiment for the synthesis of the mixed polyol citrate esters of the present disclosure.

### DETAILED DESCRIPTION

Various embodiments of the present disclosure relate to a biobased replacement for propylene glycol and ethylene glycol derived from petrochemical sources. In particular, biobased propylene glycol and ethylene glycol can be produced by hydrogenolysis of polyols derived from biological sources (i.e., bioderived). Various applications for the biobased hydrogenolysis product mixture are also disclosed. Methods of replacing petroleum derived propylene glycol and/or ethylene glycol with the biobased hydrogenolysis product mixture or biobased propylene glycol or biobased ethylene glycol are also described. The product mixture from the hydrogenolysis of bioderived polyols and the products produced therefrom may be differentiated from petroleum derived products, for example, by their carbon isotope ratios using ASTM International Radioisotope Standard Method D 6866. Products produced from the product mixture of the hydrogenolysis product from a bioderived polyol feedstock may have a 100% hiobased carbon isotope ratio. According to certain embodiments, products produced from or incorporating the product mixture of the hydrogenolysis product of the bioderived polyol feedstock may have from 1% to 99.9% biobased carbon isotope ratio. According to other embodiments, the products produced from the product mixture of the hydrogenolysis product may have from 50% to 99.9% biobased carbon isotope ratio. As used herein the term “biobased carbon isotope ratio” means a composition or component of a composition having a carbon isotope ration, as determined, for example, by ASTM International Radioisotope Standard Method D 6866, the disclosure of which is incorporated by reference herein in its entirety, that is indicative of a composition composed, in whole or in significant part, of biological products or renewable agricultural materials (including plant, animal and marine materials) or forestry materials. As used herein, the term “bioderived” means derived from or synthesized by a renewable biological feedstock, such as, for example, an agricultural, forestry, plant, bacterial, or animal feedstock.

As used in this specification and the appended claims, the articles “a”, “an”, and “the” include plural referents unless expressly and unequivocally limited to one referent.

Other than in the operating examples, or where otherwise indicated, all numbers expressing quantities of ingredients, reaction conditions and the like used in the specification and claims are to be understood as being modified in all instances by the term “about”. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference. Any material, or portion thereof that is said to be incorporated by reference herein, but which conflicts with existing definitions, statements, or other disclosure material set forth herein will only be incorporated to the extent that no conflict arises between that incorporated material and the existing disclosure material.

The present disclosure describes several different features and aspects of the invention with reference to various exemplary embodiments. It is understood, however, that the invention embraces numerous alternative embodiments, which may be accomplished by combining any of the different features and aspects described herein, in any combination that one of ordinary skill in the art would find useful.

Propylene glycol and ethylene glycol may be produced by the hydrogenolysis of various bioderived polyols (i.e., polyol feedstocks). Non-limiting examples of catalytic hydrogenolysis processes that are suitable for producing propylene glycol/ethylene glycol product mixtures for use in the various embodiments of the present disclosure may be found, for example, in U.S. Pat. Nos. 4,401,823, 5,354,914, 6,291,725 and 6,479,713, the disclosures of which are incorporated in their entirety by reference herein. As an illustrative example, the following description of the process of U.S. Pat. No. 6,479,713 is provided as one non-limiting example of a hydrogenolysis process. Substrates, such as glycerol, sorbitol, xylitol, lactic acid, arabinitol and combinations of any thereof may be subjected to hydrogenolysis over a catalyst comprising Re—Ni supported on carbon at 230° C. and 1300 psi hydrogen pressure to give two- and three-carbon glycols typically made from petrochemical-based feedstocks. In one example, after 1 hour, 25.4% glycerol conversion was achieved with 72.3% propylene glycol selectivity. According to another embodiment, the catalytic hydrogenolysis process may involve a nickel-on-alumina catalyst (commercially available from Sud-Chemie Incorporated, Louisville, Ky.) having the specifications as set forth in Table 1.

TABLE 1

| | |
|---|---|
| Nickel (wt %) | 48.9% |
| SiO₂ (wt %) | 4.59% |
| Al₂O₃ (wt %) | 30.3% |
| Shape | Cylindrical |
| Average length (mm) | 5.1 |
| Average crush strength (lbs/mm) | 1.8 |
| Reduction (%) | 43% |

The catalyst may be promoted with sodium, supplied as sodium hydroxide or sodium carbonate, to achieve a sodium loading of about 1%. The feed to this catalyst may be about 25% (w/w) sorbitol, with a specific gravity of about 1.1 g/mL and a pH of about 11.5. The reaction may be operated for up to 72 days at temperatures from about 180° C. to about 250° C. and a hydrogen pressure ranging from about 200 psi to about 1800 psi. It should be noted that the compositions and methods disclosed herein are not limited to any particular hydrogenolysis procedures, reagents, or catalysts. Rather, the compositions and methods described herein may incorporate hydrogenolysis products from polyols using any hydrogenolysis method.

Hydrogenolysis of bioderived polyol feedstocks includes polyol feedstocks derived from biological or botanical sources. For example, bioderived polyols suitable for use according to various embodiments of the present disclosure include, but are not limited to, saccharides, such as, but not limited to, biobased polyols including monosaccharides including dioses, such as glycolaldehyde; trioses, such as glyceraldehyde and dihydroxyacetone; tetroses, such as erythrose and threose; aldo-pentoses such as arabinose, lyxose, ribose, deoxyribose, xylose; keto-pentoses, such as ribulose and xylulose; aldo-hexoses such as allose, altrose, galactose, glucose (dextrose), gulose, idose, mannose, talose; keto-hexoses, such as fructose, psicose, sorbose, tagatose; heptoses, such as mannoheptulose and sedoheptulose; octoses, such as octolose and 2-keto-3-deoxy-manno-octonate; and nonoses, such as sialose; disaccharides including sucrose (table sugar, cane sugar, saccharose, or beet sugar), composed of a glucose monosaccharide moiety and a fructose monosaccharide moiety; lactose (milk sugar) composed of a glucose monosaccharide moiety and a galactose monosaccharide moiety; maltose (produced during the malting of barley) composed of two glucose monosaccharide moieties; trehalose which may be present in fungi and insects and is composed of two glucose monosaccharide moieties; cellobiose, which is another disaccharide composed of two glucose monosaccharide moieties; oligosaccharides, such as raffinose (melitose), stachycose, and verbascose; sorbitol, glycerol, sorbitan, isosorbide, hydroxymethyl furfural, polyglycerols, plant fiber hydrolyzates, fermentation products from plant fiber hydrolyzates, and various mixtures of any thereof. According to other embodiments, the bioderived polyol feedstock may be a side product or co-product from the synthesis of bio-diesel or the saponification of vegetable oils and/or animal fats (i.e., triacylglycerides).

According to certain embodiments, the bioderived polyol feedstock can be obtained by subjecting sugars or carbohydrates to hydrogenolysis (also called catalytic cracking). In one non-limiting example, sorbitol may be subjected to hydrogenolysis to provide a mixture of biobased polyols, as described herein (see, e.g. “Hydrogenolysis of sorbitol,” Clark, I., J. Ind. Eng. Chem. (Washington, D. C.) (1958), 50, 1125-6, the disclosure of which is incorporated in its entirety by reference herein). According to other embodiments, other polysaccharides and polyols suitable for hydrogenolysis include, but are not limited to, glucose (dextrose), sorbitol, mannitol, sucrose, lactose, maltose, alpha-methyl-d-glucoside, pentaacetylglucose, gluconic lactone, and combinations of any thereof (see, e.g. “Hydrogenolysis of sugars,” Zartman, W. and Adkins, H., J. Amer. Chem. Soc. (1933) 55, 4559-63, the disclosure of which is incorporated by reference herein in its entirety).

According to other embodiments, the biobased polyol feedstock may be obtained as mixed polyols. Natural fibers may be hydrolyzed (producing a hydrolyzate) to provide bioderived polyol feedstock, such as mixtures of polyols. Fibers suitable for this purpose include, but are not limited to, corn fiber from corn wet mills, dry corn gluten feed which may contain corn fiber from wet mills, wet corn gluten feed from wet corn mills that do not run dryers, distiller dry grains solubles (DDGS) and Distiller's Grain Solubles (DGS) from dry corn mills, canola hulls, rapeseed hulls, peanut shells, soybean hulls, cottonseed hulls, cocoa hulls, barley hulls, oat hulls, wheat straw, corn stover, rice hulls, starch streams from wheat processing, fiber streams from corn mesa plants, edible bean molasses, edible bean fiber, and mixtures of any thereof. Hydrolyzates of natural fibers, such as corn fiber, may be enriched in bioderived polyol feedstock suitable for use as a feedstock in the hydrogenation reaction described herein, including, but not limited to, arabinose, xylose, sucrose, maltose, isomaltose, fructose, mannose, galactose, glucose, and mixtures of any thereof.

According to other embodiments, the bioderived polyol feedstock obtained from hydrolyzed fibers may be subjected to fermentation. The fermentation process may provide new bioderived polyol feedstocks, or may alter the amounts of residues of polysaccharides or polyols obtained from hydrolyzed fibers. After fermentation, a fenestration broth may be obtained and residues of polysaccharides or polyols can be recovered and/or concentrated from the fermentation broth to provide a bioderived polyol feedstock suitable for hydrogenolysis, as described herein.

Hydrogenolysis of a bioderived polyol feedstocks, for example, any of the bioderived polyol feedstocks set forth herein, results in a hydrogenolysis product. As used herein, the terms “hydrogenolysis product” and “hydrogenolysis product mixture” are synonymous and may be used interchangeably According to certain embodiments of the present disclosure, the hydrogenolysis product comprises a mixture of propylene glycol and ethylene glycol containing minor amounts of one or more of methanol, 2-propanol, glycerol, lactic acid, glyceric acid, sodium lactate, butanediols, and sodium glycerate. As used herein, the term “butanediols” include 1,2-butanediol, 1,3-butanediol, 1,4-butanediol, 2,3-butanediol, and mixtures of any thereof. According to certain embodiments, the hydrogenolysis product may comprise 0.1% to 99.9% by weight of propylene glycol, 0.1% to 99.9% by weight of ethylene glycol, 0% to 99.9% by weight of methanol, 0% to 99.9% by weight of 2-propanol, 0% to 99.9% by weight of glycerol, 0% to 99.9% by weight of lactic acid, 0% to 99.9% by weight of glyceric acid, 0% to 99.9% by weight of butanediols, 0% to 99.9% by weight of sodium lactate, and 0% to 99.9% by weight of sodium glycerate. The composition of the hydrogenolysis product mixture may be dependent on certain conditions, such as, for example, the particular bioderived polyol feedstock or the hydrogenolysis process used. For example, mixed polyols may be synthesized by feeding a 25% sorbitol solution into a reactor containing an aluinina-based massive nickel catalyst (cylinder shaped) promoted with sodium hydroxide or sodium carbonate to 1% sodium. Over a period of 72 days, the feed (specific gravity=1.1 g/mL, pH˜11.5) may be fed into the reactor held at a temperature of 180° C. to 250° C. under from 200 psi to 1800 psi pressure. A representative product contained 47% propylene glycol, 20% ethylene glycol, 21% glycerol, and the remainder was mixed diols.

The composition comprising the hydrogenolysis product, as set forth herein, may have a 100% biobased carbon isotope ratio as determined by ASTM International Radioisotope Standard Method D 6866. The composition mnay be differentiated from, for example, similar compositions comprising petroleum derived components by comparison of the carbon isotope ratios, for example, the ¹⁴C/¹²C or the ¹³C/¹²C carbon isotope ratios, of the two compositions. As described herein, isotopic ratios may be determined, for example, by liquid scintillation counting, accelerator mass spectrometry, or high precision isotopic ratio mass spectrometry.

According to certain embodiments, the hydrogenolysis product comprising a mixture of propylene glycol and ethylene glycol, along with minor amounts of one or more of methanol, 2-propanol, glycerol, lactic acid, glyceric acid, butanediols, sodium lactate, and sodium glycerate, may be used as a replacement for propylene glycol and/or ethylene glycol that has been derived or synthesized from petrochemical or petroleum derived products. As used herein, the term “replacement,” when used in the context of the hydrogenolysis product replacing petroleum derived propylene glycol and/or ethylene glycol, means that during a manufacturing or formulation process, petroleum derived propylene glycol and/or ethylene glycol ingredients are omitted from the process and the hydrogenolysis product mixture or component thereof or a composition produced from the hydrogenolysis product mixture is added in place of the petroleum derived glycols. The hydrogenolysis product mixture or component thereof may be added to the formulation in the same amount (on a molar bases, such as moles of hydroxyl moieties) as the petroleum derived glycol or, alternatively, a greater or lesser amount of the hydrogenolysis may be used relative to the amount of petroleum derived glycol being replaced. When used as a replacement for petrochemical or petroleum derived propylene glycol and/or ethylene glycol, the hydrogenolysis product mixture may provide a low cost substitute or replacement for the petroleum based propylene glycol/ethylene glycol with the added benefit that the replacement is derived from a renewable biological resource. According to certain embodiments, the hydrogenolysis product may be used as a complete replacement for propylene glycol and/or ethylene glycol that has been derived or synthesized from petrochemical or petroleum derived products (i.e., the hydrogenolysis product replaces 100% of the petroleum derived propylene glycol and/or ethylene glycol). According to other embodiments, the hydrogenolysis product may be used as a partial replacement for propylene glycol and/or ethylene glycol that has been derived or synthesized from petrochemical or petroleum derived products (i.e., the hydrogenolysis product replaces from 1% to 99.9% of the petroleum derived propylene glycol and/or ethylene glycol). According to still other embodiments, the hydrogenolysis product may replace from 50% to 100% of the petroleum derived propylene glycol and/or ethylene glycol. According to still other embodiments, the hydrogenolysis product may replace from 70% to 100% of the petroleum derived propylene glycol and/or ethylene glycol, According to still other embodiments, the hydrogenolysis product may replace from 90% to 100% of the petroleum derived propylene glycol and/or ethylene glycol.

According to certain embodiments, wherein the composition comprising the hydrogenolysis product mixture, as described herein, may be used as the replacement for petroleum derived propylene glycol and/or ethylene glycol, the hydrogenolysis product mixture may be used directly as a replacement for the petroleum derived glycols. That is, the hydrogenolysis product mixture may be used as a mixture of propylene glycol and ethylene glycol, along with minor amounts of one or more of methanol, 2-propanol, glycerol, lactic acid, glyceric acid, butanediols, sodium lactate, and sodium glycerate, without substantial purification or purification into the components of the mixture. Thus, for example, in a formulation mixture that typically comprises petroleum derived propylene glycol, the petroleum derived propylene glycol may be directly replaced, in part or completely, with the hydrogenolysis product mixture. Similarly, in a formulation mixture that typically comprises petroleum derived ethylene glycol, the petroleum derived ethylene glycol may be directly replaced, in part or completely, with the hydrogenolysis product mixture.

In other embodiments, the hydrogenolysis product mixture may be at least partially purified by a purification method prior to being used as a replacement, either total or partial, for petroleum derived glycols. As used herein, the phrase “at least partially purified” means that at least a portion of at least one of the components of the composition has been separated from at least a portion of at least one other component of the composition by a purification method. The hydrogenolysis product mixture may be at least partially purified by a purification method selected from the group consisting of chromatography, such as, for example, ion exclusion chromatography, ion exchange chromatography, simulated moving bed chromatography, liquid chromatography, and gas chromatography; extraction, such as, for example, acid/base extraction; electrodialysis; and distillation, such as, for example, simple distillation, fractional distillation, steam distillation, reduced pressure or vacuum distillation, continuous distillation, batch distillation, extractive distillation, azeotropic distillation, and combinations of any thereof. According to certain embodiments, the at least partially purified hydrogenolysis product may be used as a replacement for petroleum derived propylene glycol or ethylene glycol. In certain applications, it may be desirable for the hydrogenolysis product to be at least partially purified prior to being used as a replacement for petroleum derived propylene glycol or ethylene glycol, for example, in applications where removal of acidic components (ie., lactic acid and/or glyceric acid), ionic components (i.e., sodium lactate, sodium glycerate and/or residual ionic salts), and/or monohydroxyl components (i.e., methanol and/or 2-propanol) may be desired.

Propylene glycol may be a component in certain latex paint formulations. For example, in 2003, 37 million pounds of propylene glycol were used in the production of latex paint. According to certain latex paint formulations, the propylene glycol may be used as a humectant. Currently, the propylene glycol used in latex paint formulations is derived from petrochemical sources. According to certain embodiments, the hydrogenolysis product mixture of the present disclosure may be used as a replacement of at least a portion of or all of the petroleum derived propylene glycol in certain latex paint formulations.

According to various embodiments, the hydrogenolysis product mixture may be used as a direct replacement for petroleum derived propylene glycol in a latex paint formulation. For example according to certain embodiment, using the hydrogenolysis mixture as a direct replacement for petroleum derived propylene glycol may result in lower manufacturing costs, since the hydrogenolysis mixture may be a less expensive feedstock (i.e., less expensive that petroleum derived propylene glycol). According to other embodiments, the hydrogenolysis product mixture may be at least partially purified prior to being used as a replacement for petroleum derived propylene glycol in a latex paint formulation (i.e., prior to adding the hydrogenolysis product to the latex paint formulation). For example, for certain latex paint formulations, it may be desirable to remove one or more components of the hydrogenolysis product mixture, such as, for example, methanol and/or 2-propanol, prior to adding the hydrogenolysis product to the latex paint formulation. Certain latex paint formulations may comprise from 2% to 15% by volume of petroleum derived propylene glycol. At least some, and in certain embodiments all, of the petroleum derived propylene glycol may be replaced by the hydrogenolysis product, which according to certain embodiments may have been at least partially purified. Thus, according to certain embodiment the present disclosure may include a latex paint formulation comprising from 2% to 15% by volume of the hydrogenolysis product which in certain embodiments, may have been at least partially purified. Alternatively or in addition to replacing a petroleum derived propylene glycol with the hydrogenolysis mixture, a fatty acid ester product of the hydrogenolysis mixture, for example, a mixture comprising a propylene glycol mono ester product from a hydrogenolysis mixture, may be added as a component of a latex paint formulation, for example, as a replacement of a petroleum derived propylene glycol mono ester product.

Petroleum derived ethylene glycol and/or propylene glycol may also be used as components of de-icing and/or antifreeze formulations. Formulations comprising petroleum derived propylene glycol and/or ethylene glycol have been used for such applications as de-icing of airplanes and roads, as well as various antifreeze and industrial coolant applications. Approximately 26% of all petroleum derived ethylene glycol produced is used in antifreeze formulations. Approximately 20% of petroleumn derived propylene glycol produced is used in de-icing and antifreeze formulation. In addition, a switch to the use of petroleum derived propylene glycol instead of petroleum derived ethylene glycol in de-icing and antifreeze application has begun to occur due to toxicity concerns connected with ethylene glycol.

According to certain embodiments, the biobased hydrogenolysis product mixture described herein may be used as an at least partial replacement for petroleum derived propylene glycol and/or ethylene glycol in de-icing and/or antifreeze formulations. In addition to the de-icing and low freezing capabilities of the bioderived propylene glycol/ethylene glycol of the hydrogenolysis product mixture, the unpurified mixture may further benefit from a freezing point depression resulting from the minor components of the mixture resulting from the hydrogenolysis process (i.e., methanol, 2-propanol, glycerol, lactic acid, glyceric acid, butanediols, and acid salts), when compared to pure petroleum derived propylene glycol de-icing/antifreeze compositions. As used herein, the term “freezing point depression” is a colligative property of a solution where the temperature at which the solution freezes is lowered (relative to the pure solvent) due to the presence of solutes, such as impurities. Thus, in addition to the benefits of the biobased hydrogenolysis product mixture, described herein, use of the hydrogenolysis product mixture in de-icing and/or antifreeze formulations may also be more effective and may be used in lower quantities than petroleum derived propylene glycol or ethylene glycol in comparable de-icing and/or antifreeze formulations. For example, in applications where the hydrogenolysis product mixture is used in a road de-icing formulation (i.e., to prevent or reduce build-up or formation of ice on roadways), use of the unpurified hydrogenolysis product mixture, which may include salts, such as, sodium lactate and/or sodium glycerate, may be desired, for example to reduce both the cost and amount of de-icing formulation required. Alternatively, when used as an antifreeze formulation or industrial coolant, or in an aeronautical de-icing formulation, use of an at least partially purified hydrogenolysis product mixture, for example a mixture where salts have been removed, may be desired. According to certain embodiments, the present disclosure provides for a de-icing formulation or antifreeze formulation comprising a composition having a 100% biobased carbon isotope ratio. According to other embodiments, the present disclosure provides for a de-icing formulation or antifreeze formulation comprising a composition having from 50% to 100% biobased carbon isotope ratio. That is, in various embodiments, from 50% up to all of petroleum derived propylene glycol and/or ethylene glycol may be replaced with the hydrogenolysis product composition of the present disclosure

According to other embodiments, the hydrogenolysis product mixture of the bioderived polyol may be used as a monomer in a polymerization reaction. For example, the hydrogenolysis product mixture comprising a mixture of propylene glycol and ethylene glycol, along with minor amounts of one or more of methanol, 2-propanol, glycerol, lactic acid, glyceric acid, butanediols, sodium lactate, and sodium glycerate may be used as a replacement for petroleum derived propylene glycol and/or ethylene glycol as a diol monomer reagent in a polyester polymerization reaction, In certain embodiments the hydrogenolysis product mixture may be used as a replacement for petroleum derived propylene glycol and/or ethylene glycol as a diol monomer reagent in a polymerization reaction to make an unsaturated polyester.

Polyester polymers are polymers resulting from the condensation polymerization reaction of a diol monomer reagent and a dicarboxylic acid or dicarboxylic acid derivative monomer reagent. The resulting polymer comprises of a series of alternating diol and dicarboxylic acid monomer units linked together by ester linkages formed from the condensation of a hydroxyl group on the diol monomer reagent and a carboxylic acid group (or carboxylic acid derivative) on the dicarboxylic acid monomer reagent. Petroleum derived ethylene glycol and propylene glycol are major feedstocks for the industrial synthesis of polyester polymers, such as, for example, polyethylene terephthalate, and other polyester resins. The polyesters resulting from petroleum derived monomer reagents will have a carbon isotope ratio characteristic of petroleum derived carbon material.

According to certain embodiments, the hydrogenolysis product mixture described herein may be used as an at least partial replacement for a petroleum derived propylene glycol or a petroleum derived ethylene glycol as the diol monomer reagent in a polyester polymer synthesis reaction. The resulting polyester polymer may have a composition having, at least in part, a biobased carbon isotope ratio. For example, when the diol monomer reagent comprising the biobased hydrogenolysis product mixture is reacted with a petroleum derived dicarboxylic acid monomer reagent, the resulting polyester will have a diol monomer component having a biobased carbon isotope ratio and a dicarboxylic acid monomer component having a carbon isotope ratio associated with a petroleum derived product. Alternatively, the resulting polyester polymer may have a composition having a 100% biobased carbon isotope ratio when the diol monomer reagent comprising the biobased hydrogenolysis product mixture is reacted with a bioderived dicarboxylic acid (or bioderived dicarboxylic acid derivative) monomer reagent.

Thus, according to certain embodiments, the diol monomer reagent comprising the biobased hydrogenolysis product mixture, wherein the hydrogenolysis product mixture comprises a mixture of propylene glycol and ethylene glycol, along with, optionally, minor amounts of one or more of methanol, 2-propanol, glycerol, lactic acid, glyceric acid, butanediols, sodium lactate, and sodium glycerate, may be reacted with a dicarboxylic acid monomer reagent, such as those selected from the group consisting of a petroleum derived saturated dicarboxylic acid or dicarboxylic acid derivative monomer reagent, a petroleum derived unsaturated dicarboxylic acid or dicarboxylic acid derivative monomer reagent, a bioderived saturated dicarboxylic acid or dicarboxylic acid derivative monomer reagent, a bioderived dicarboxylic acid or dicarboxylic acid derivative monomer reagent, or a mixture of any thereof

In various embodiments where the diol monomer reagent comprising the hydrogenolysis product mixture is reacted with a petroleum derived dicarboxylic acid monomer reagent, the petroleum derived dicarboxylic acid, which may be saturated or unsaturated, may be any petroleum derived carboxylic acid that may be typically used in polyester formation. Suitable non-limiting examples of petroleum derived dicarboxylic acids monomer reagents include terephthalic acid, isophthalic acid, phthalic acid, diphenyl-p,p′-dicarboxylic acid, naphthalene-2,7-dicarboxylic acid, naphthalene-2,6-dicarboxylic acid, diphenylmethane-p,p′-dicarboxylic acid, benzophenone-4,4′-dicarboxylic acid, 1,2-diphenoxymethane-p,p′-dicarboxylic acid, maleic acid, glutaric acid, cyclolhexane-dicarboxylic acid, succinic acid, malonic acid, adipic acid, mesaconic acid, itaconic acid, citraconic acid, sebacic acid, fumaric acid, and mixtures of any thereof, including acyl halides, anhydrides and esters of all the above acids.

In specific embodiments where the diol monomer reagent comprising the hydrogenolysis product mixture may be reacted with a bioderived unsaturated dicarboxylic acid monomer reagent, the bioderived unsaturated dicarboxylic acid may be fumaric acid, such as, for example, fumaric acid derived from a fermentation process; 2,5-furandicarboxylic acid, such as, for example, 2,5-furandicarboxylic acid derived from fructose; a C₁₈- to C₂₄-unsaturated dicarboxylic acid, such as, for example, a C₁₈- to C₂₄-unsaturated dicarboxylic acid produced by any of the methods disclosed in U.S. Pat. No. 6,569,670 to Anderson et al., and D. L,. Craft et al., Applied and Environmental Microbiology, (2003) 69(10), 5983-5991, the disclosures of which are incorporated in their entirety by reference herein; a dimerized unsaturated fatty acid; other polymerized fatty acids; dicarboxylic acid esters; polycarboxylic acids, such as those produced by heat-bodying oils and hydrolysis of ester bonds; or various mixtures of any thereof. In certain embodiments, 2,5-furan dicarboxylic acid or monoalkyl, dialkyl or polyalkyl esters of polycarboxylic acids (including dicarboxylic acids) may be employed to operate at lower temperatures.

In other embodiments where the diol monomer reagent comprising the hydrogenolysis product mixture may be polymerized with a bioderived saturated dicarboxylic acid monomer reagent, the bioderived saturated dicarboxylic acid may be succinic acid, such as, for example, succinic acid derived from a fermentation process; tetrahydrofuran-2,5-dicarboxylic acid, such as, for example, tetrahydrofuran-2,5-dicarboxylic acid derived from fructose; a C₁₈- to C₂₄-saturated dicarboxylic acid, such as, for example, a C₁₈- to C₂₄-saturated dicarboxylic acid produced by any of the methods disclosed in U.S. Pat. No. 6,569,670 to Anderson et al., and D. L. Craft et al., Applied and Environmental Microbiology, (2003) 69(10), 5983-5991; a dimerized saturated fatty acid; saturated polycarboxylic acids of hydrogenated heat-bodied oils, saturated dimer and trimer fatty acids; or various mixtures of any thereof.

According to other embodiments, the diol monomer reagent comprising the hydrogenolysis product mixture may be further mixed with at least one other biobased diol monomer reagent prior to polymerization with the dicarboxylic acid or dicarboxylic acid derivative monomer reagent. For example, according to certain embodiments the hydrogenolysis product may be mixed with one or more other biobased diol monomer reagent selected from the group consisting of tetrahydro-2,5-furandimethanol, derived from fructose; 2,5-furandimethanol, derived from fructose; 1,3-propanediol, such as, for example, biomass derived 1,3-propanediol; an octadecanediol, such as, for example, 1,18-octadecanediol, 1,9-octadecanediol, or 1,10-octadecanediol; a fatty alcohol dimer, such as, a fatty alcohol dimer made from a bioderived fatty acid dimer; isosorbide; isomannide; and various mixtures of any thereof. Other useful polyols may include, but are not limited to: sorbitol, mannitol, polyglycitol, maltitol, xylitol, lactitol, isomalt, erythritol, glycerol, and mixtures of any thereof. The resulting mixture of the hydrogenolysis product mixture and the second biobased diol monomer reagent may then be polymerized with a dicarboxylic acid monomer reagent, as described herein, to form a polyester polymer. The resulting polyester polymer may have different properties than the polymer made from using only the hydrogenolysis product as the diol component.

In other embodiments, the bioderived hydrogenolysis product mixture may also be at least partially purified, as described herein, to afford an at least partially purified hydrogenolysis product, for example, a purified hydrogenolysis product consisting substantially of, and in some embodiments consisting essentially of, propylene glycol or a purified hydrogenolysis product consisting substantially of, and in some embodiments consisting essentially of, ethylene glycol prior to mixing with the dicarboxylic acid monomer component. According to these embodiments, the at least partially purified hydrogenolysis product may then be used as the diol monomer reagent in a condensation polymerization reaction to form a polyester with a dicarboxylic acid (or dicarboxylic acid derivative) monomer reagent, as set forth herein.

In any of the condensation polymerization reactions described herein, yielding polyesters having at least a partial, and in certain embodiments 100%, biobased carbon isotope ratio described herein, the polyester polymerization reaction may further comprise a modifier, such as, for example, a modifier derived from a petroleum derived material or a biobased material. Modifiers may be added to the polymerization reaction, for example, to adjust the properties of the resulting polymer or to change the polymerization process. For example, certain modifiers may act as additives to the polymerization reaction where the resulting polymer incorporates the modifier additive. Alternatively, other modifiers may act as cross-linkers, thereby cross-linking adjacent polymer strands within the resultant polymer. In certain embodiments comprising modifiers where all the organic components of the polymerization (i.e., the diol reagent, the dicarboxylic acid reagent, and modifier) are biobased, the resulting polyester polymer will have a 100% biobased carbon isotope ratio. Alternatively, polyesters with a biobased carbon isotope ratio that is less than 100% may be synthesized by reacting the hydrogenolysis product as the diol monomer with a dicarboxylic acid reagent and a modifier, wherein at least one of the dicarboxylic acid reagent and the monomer are derived from a petroleum product.

According to certain embodiments where a bioderived modifier is used in the polymerization reaction, non-limiting examples of the bioderived modifier may include, furfural derivatives, such as, for example, a 2,5-dihydroxymethylfurfural derivative or a 5-hydroxymethylfurfural derivative, or diether derivatives of 2,5-dihyroxylmethylfurfural or 5-hydroxymethylfurfural with either propylene glycol, ethylene glycol, or the hydrogenolysis product mixture of the present disclosure; and difurfuryl ether.

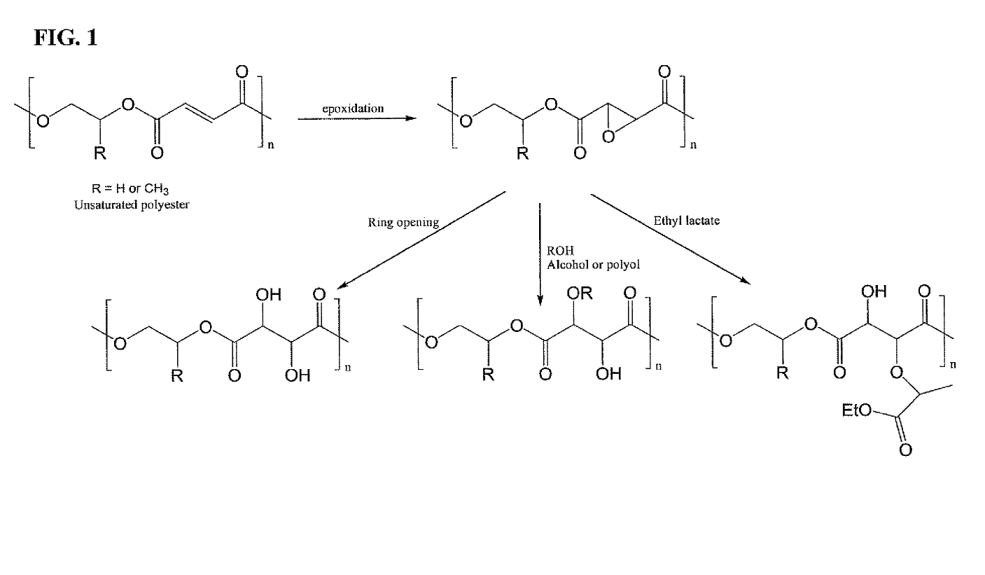

According to various embodiments where an unsaturated polyester is formed from the hydrogenolysis product mixture, for example, when the hydrogenolysis product mixture is reacted with an unsaturated dicarboxylic acid monomer reagent, as described herein, the resulting unsaturated polyester may be further derivatized or modified by one or more chemical reactions. FIG. 1 shows one non-limiting general reaction scheme for the modification of a bioderived unsaturated polyester according to the present disclosure. For example, and with reference to FIG. 1, according to certain embodiments, one or more double bonds in the resulting unsaturated polyester may be epoxidized using either chemical or enzymatic means. The resulting epoxy polyester may have desired properties as a product of manufacture or, alternatively, may be further reacted to yield further modified polyesters. In examples of further modification according to certain non-limiting embodiments, some or all of the epoxide rings of the epoxidized polyester may be opened, for example, by hydrolysis to give a polyester having vicinal diol moieties, or by reaction with a bioderived alcohol or polyol to give a polyester having 1,2-hydroxyether moieties. The resulting modified polyesters may have applications, for example, as polyurethane-polyols, or as metal chelating polymers for water treatment or mining operations. According to certain embodiments, the resulting modified polyer may still have a 100% bioderived carbon isotope ratio. Alternatively, if the reagent used to open the epoxide ring of the polyester is derived from a petroleum source or other non-biobased source, the resulting modified polymer will have a bioderived carbon isotope ratio of less than 100%.

According to other embodiments of the present disclosure, the composition comprising a hydrogenolysis product mixture of a bioderived polyol may be used as a replacement for petroleum derived propylene glycol and/or ethylene glycol as the alcohol component in the synthesis of bioderived esters, such as, for example, fatty acid esters including propylene glycol monoesters (“PGMEs”) and propylene glycol diesters; levulinate esters; mixed polyol lactate esters; and mixed polyol citrate esters. The resulting bioderived esters may have a 100% biobased carbon isotope ratio, for example, when the fatty acid, levulinic acid, and lactic acid components are also bioderived.

The esters produced from petroleum derived propylene glycol and fatty acid methyl esters or triglycerides (i.e., PGME) have multiple industrial uses, such as, for example, as coalescents in latex paints and other formulations. Examples of a latex paint coalescent comprising a petroleum derived PGME include Archer RCS (a registered trademark of Archer Daniels Midland Company, Decatur, Ill.). According to certain embodiments, the hydrogenolysis product may be used as a replacement for petroleum derived propylene glycol and/or petroleum derived ethylene glycol in the synthesis of PGMEs.

According to various embodiment, the hydrogenolysis product mixture comprising a mixture of propylene glycol and ethylene glycol, along with minor amounts of one or more of methanol, 2-propanol, glycerol, lactic acid, glyceric acid, butanediols, sodium lactate, and sodium glycerate may react as a reagent in the synthesis of a PGME having a 100% biobased carbon isotope ratio. For example, in certain embodiments, the hydrogenolysis product mixture may be reacted with either a bioderived fatty acid or fatty acid ester, such as a methyl ester, or a bioderived glyceride, such as a mono glyceride, a diglyceride, and/or a triglyceride, to form a bioderived PGME via an esterification or transesterification reaction. Typically, it is desirable to synthesize a PGME mixture which possesses a high ratio of monoester to diester. Examples of methods for the preparation of PGME having a high monoester:diester ratio which are suitable for use with the hydrogenolysis product mixtures of the present disclosure may be found in U.S. Pat. No. 6,723,863, the disclosure of which is incorporated in its entirety by reference herein.

In particular, as described in U.S. Pat. No. 6,723,863, reacting the hydrogenolysis product mixture with a triglyceride, such as a vegetable oil or animal fat, in the presence of a catalyst at a temperature ranging from 180° C. to 280° C. under an inert atmosphere at a pressure from 0 to 500 psig will produce a propylene glycol monoester mixture having a high monoester:diester ratio. According to certain embodiments, the hydrogenolysis product mixture may be reacted with a triglyceride, such as those selected from the group consisting of corn oil, soybean oil, canola oil, vegetable oil, safflower oil, sunflower oil, nasturtium seed oil, mustard seed oil, olive oil, sesame oil, peanut oil, cottonseed oil, rice bran oil, babassu nut oil, castor oil, palm oil, palm kernel oil, rapeseed oil, low erucic acid rapeseed oil, lupin oil, jatropha oil, coconut oil, flaxseed oil, evening primrose oil, jojoba oil, tallow, beef tallow, butter, chicken fat lard, dairy butterfat, shea butter, biodiesel, used frying oil, oil miscella, used cooking oil, yellow trap grease, hydrogenated oils, derivatives of these oils, fractions of these oils, conjugated derivatives of these oils and mixtures of any thereof.

According to other embodiments, the hydrogenolysis product mixture may be reacted with either bioderived fatty acids or fatty acid esters, such as a methyl esters, or a bioderived glyceride, such as a monoglyceride, a diglyceride, and/or a triglyceride, to form a bioderived propylene glycol diester.

According to various embodiments, the PGMEs having 100% biobased carbon isotope content, for example when the PGMEs synthesized from the hydrogenolysis product of the various embodiments of the present disclosure, may be used in a candle wax formulation. According to certain embodiments, the present disclosure includes a candle wax formulation comprising a PGME composition synthesized from a hydrogenolysis product mixture as described herein. The hydrogenolysis product may be used as a replacement for petroleum derived propylene glycol and/or ethylene glycol in the synthesis of the PGME product. In still other embodiments, the PGMEs may be used in the production of specialty esters for coalescents, wax modifiers, such as candle modifiers, lubricants, or drilling fluids, In still other embodiments, the hydrogenolysis product mixture may be esterified with levulinic acid to produce levulinate esters

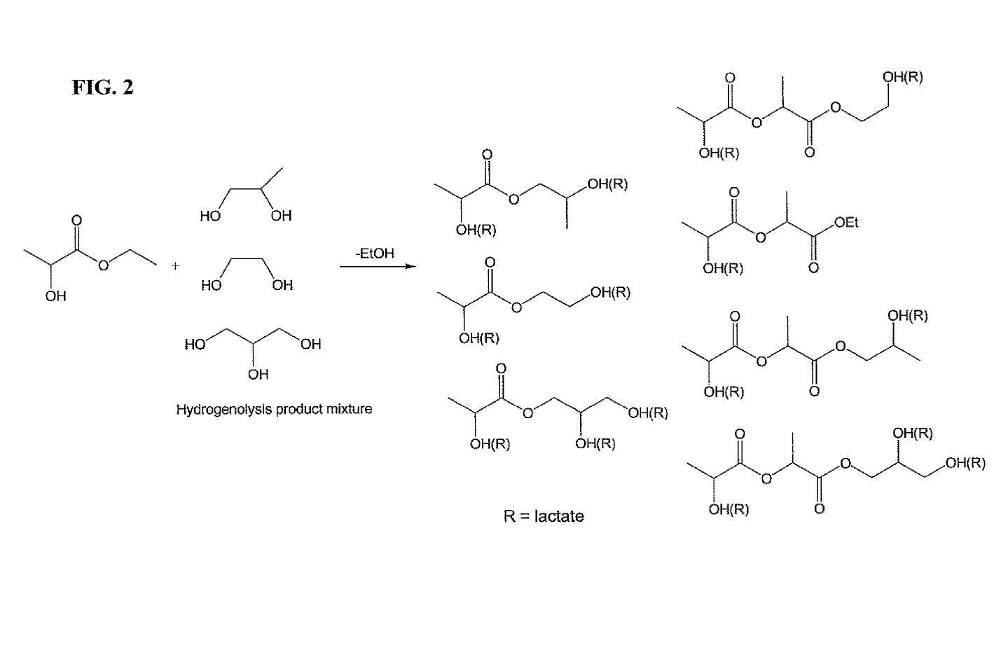

Ethyl lactate and other lactate esters may be used as solvents. However, their use as solvents may have certain drawbacks, such as, high volatility, low flashpoints and/or unpleasant odor. According to certain embodiments, the hydrogenation product of the bioderived polyol feedstock, as described herein, may be reacted with biobased lactic acid or a biobased lactic acid derivative, such as a lactate ester, to produce a mixed polyol lactate ester composition. Lactate esters synthesized by esterification (with lactic acid) or transesterification (for example, with ethyl lactate or other alkyl lactate) with the glycerol, propylene glycol and/or ethylene glycol of the hydrogenolysis product mixture will produce a mixed polyol lactate ester composition. The mixed polyol lactate ester composition may benefit from a decrease in the unpleasant odor, as well as a decrease in volatility and increase in flashpoint when compared to simple lactate ester solvents, and thus may have benefits compared to existing lactate ester solvents. Alternatively, the mixed polyol lactate ester may be used for certain polymer applications, such as, for example, as a polyol component in a polyurethane polymerization reaction. In addition, when the lactic acid or lactate ester is bioderived, the resulting mixed polyol lactate ester composition may have a 100% biobased carbon isotope ratio. FIG. 2 illustrates a reaction scheme for the synthesis of a mixed polyol lactate ester composition, according to one embodiments of the present disclosure. The mixed polyol lactate ester compositions may be used as an industrial solvent, for example, as a degreasing solvent, and may have a higher polarity than typical lipophilic solvents such that the composition may be water soluble.

Referring again to FIG. 2, a mixed polyol lactate ester composition having 100% biobased carbon isotope ratio may be synthesized by reacting the hydrogenolysis product mixture with ethyl lactate in a transesterification reaction. The reaction may be forced to completion, for example, by the removal of ethanol. According to certain embodiments, a mixture of polyol lactate esters may be produced comprising, in part, 2-hydroxypropyl lactate, 2-hydroxyethyl lactate, 2,3-dihydroxypropyl lactate, 2-hydroxylethyl 2-(lactoyloxy)propanoate, ethyl 2-(lactoyloxy)propanoate, 2-hydroxypropyl 2-(lactoyloxy)propanoate, and 2,3-dihydroxypropyl 2-(lactoyloxy)propanoate, and mixtures of any thereof, along with other components, such as, for example, structures where the free hydroxyl groups of the structures listed above have been esterified with excess lactic acid to form additional lactate esters. In light of the disclosure herein, one skilled in the art will recognize that the above mixed polyol lactate ester composition of FIG. 2 is presented for illustration purposes only and is in no way limiting of the mixed polyol lactate ester composition, and that further complex mixtures may be made depending on the composition of the hydrogenolysis product mixture and the quantity of ethyl lactate. Further, one skilled in the art will recognize that the percent composition of the various polyol lactate ester components may vary depending on the compositional make-up of the hydrogenolysis product mixture (i.e., the relative percentages of propylene glycol, ethylene glycol and other components in the hydrogenolysis product mixture), the amount of lactate starting material, and the particular reaction conditions used.

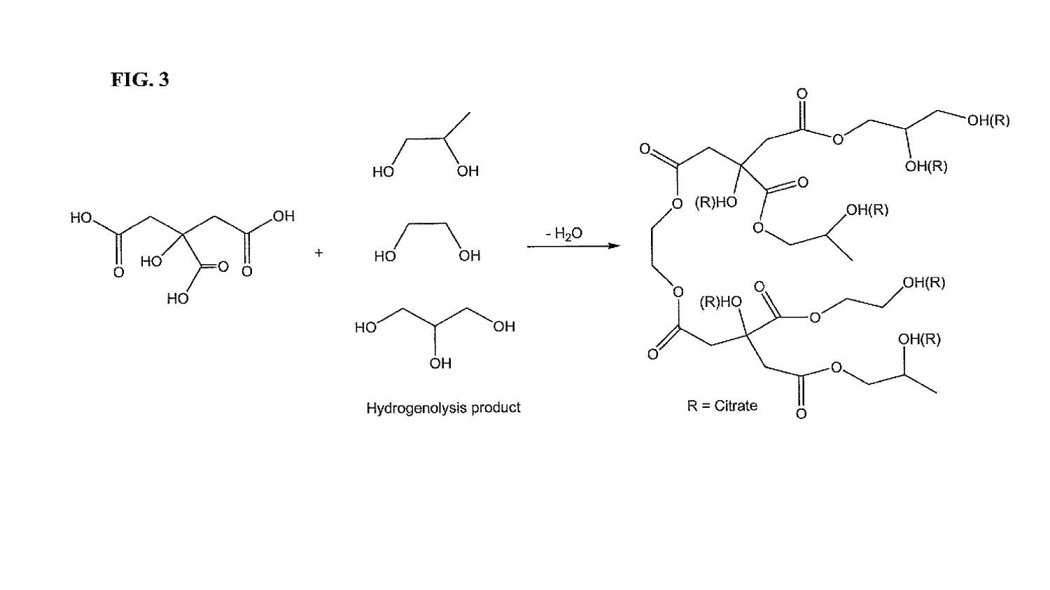

According to other embodiments, the hydrogenation product of the bioderived polyol feedstock, as described herein, may be reacted with biobased citric acid or a biobased citric acid derivative, such as a citrate ester (for example, triethyl citrate), to produce a mixed polyol citrate ester composition. The mixed polyol citrate ester composition may comprise oligomeric polyester-polyols, as described below, which may be used, for example, in polyurethane applications, such as a polyol component in a two component polyurethane composition, for example in a high molecular weight polyurethane, or as a plasticizer in a polyurethane composition. For example, the mixed polyol citrate ester composition may be reacted with a diisocyanate component to form a two component polyurethane. In addition, when the citric acid or citrate ester is bioderived, the resulting mixed polyol citrate ester composition may have a 100% biobased carbon isotope ratio. Where the diisocyanate is also derived from a biological source material, the resulting polyurethane may have a 100% bioderived carbon isotope ratio. Alternatively according to other embodiments, the mixed polyol citrate ester may be used as a chelating agent, for example as a metal chelating agent. FIG. 3 illustrates a reaction scheme for the synthesis of a mixed polyol citrate ester composition, according to one embodiments of the present disclosure.

Referring again to FIG. 3, a mixed polyol citrate ester composition having 100% biobased carbon isotope ratio may be synthesized by reacting the hydrogenolysis product mixture, which may also contain residual or additional glycerol, with biobased citric acid in an esterification reaction. The reaction may be forced to completion, for example, by the removal of water. According to certain embodiments, a mixture of polyol citrate esters may be produced, including the mixed polyol citrate ester set forth in FIG. 3, along with other components, for example, structures where the free hydroxyl groups of the structures listed herein have been further esterified with excess citric acid. In light of the disclosure herein, one skilled in the art will recognize that the above mixed polyol citrate ester composition of FIG. 3 is presented for illustration purposes only and is in no way limiting of the mixed polyol citrate ester composition, and that further complex mixtures may be made depending, for example, on the composition of the hydrogenolysis product mixture or quantity of citric acid used. Further, one skilled in the art will recognize that the percent composition of the various polyol citrate ester components may vary depending on the compositional make-up of the hydrogenolysis product mixture (i.e., the relative percentages of propylene glycol, ethylene glycol and other components in the hydrogenolysis product mixture), the amount of citric acid starting material, and the particular reaction conditions used.

According to still other embodiments, the present disclosure also contemplates various methods of making a biobased composition to be used as a replacement for petroleum derived propylene glycol or ethylene glycol. According to one embodiment, the method may comprise reacting a bioderived polyol feedstock selected from the group consisting of glucose, sorbitol, glycerol, sorbitan, isosorbide, hydroxymethyl furfaral, a polyglycerol, plant fiber hydrolyzates, fermentation products from plant fiber hydrolyzates, and mixtures of any thereof, via a hydrogenolysis process, as set forth herein, to give a hydrogenolysis product mixture. The hydrogenolysis product mixture may comprise a mixture of propylene glycol, ethylene glycol, and one or more of methanol, 2-propanol, glycerol, lactic acid, glyceric acid, butanediols, sodium lactate, and sodium glycerate. The resulting hydrogenolysis product mixture may comprise components that are 100% biobased. The method further comprises the step of adding the hydrogenolysis product to a formulation as a replacement for petroleum derived propylene glycol and/or ethylene glycol.

According to certain embodiments of the methods, adding the hydrogenolysis product mixture to a formulation may comprise adding the hydrogenolysis product mixture to a latex paint formulation as a replacement, at least in part, of petroleum derived propylene glycol and/or ethylene glycol. According to certain embodiments, the hydrogenolysis product mixture may be added as a complete replacement for petroleum derived propylene glycol and/or ethylene glycol (i.e., as a replacement for 100% of the petroleum derived propylene and ethylene glycol). According to other embodiments, the hydrogenolysis product mixture may be added as a partial replacement for petroleum derived propylene glycol and/or ethylene glycol. In certain embodiments, the hydrogenolysis product mixture may be added to replace from 50% to 99.9% of petroleum derived propylene glycol and/or ethylene glycol. According to still other embodiments, the method may further comprise the step of at least partially purifying the hydrogenolysis product mixture prior to adding the hydrogenolysis product to a formulation, such as, for example, a latex paint formulation. In certain embodiments, the step of at least partially purifying the hydrogenolysis product mixture may comprise at least partially purifying the hydrogenolysis product mixture using any suitable purification process, such as those processes discussed herein.

According to other embodiments of the methods, adding the hydrogenolysis product mixture to a formulation may comprise adding the hydrogenolysis product mixture to a de-icing or antifreeze formulation as a replacement, at least in part, for petroleum derived propylene glycol and/or ethylene glycol, According to certain embodiments, the hydrogenolysis product mixture is added as a complete replacement for petroleum derived propylene glycol and/or ethylene glycol (i.e., as a replacement for 100% of the petroleum derived propylene and ethylene glycol). According to other embodiments, the hydrogenolysis product mixture may be added as a partial replacement for petroleum derived propylene glycol and/or ethylene glycol. In certain embodiments, the hydrogenolysis product mixture may be added to replace from 50% to 100% of petroleum derived propylene glycol and/or ethylene glycol.

According to other embodiments, the present disclosure contemplates methods for making a bioderived polyester polymer. The method may comprise the steps of mixing a hydrogenolysis product with one of a bioderived saturated dicarboxylic acid monomer reagent and an unsaturated dicarboxylic acid monomer reagent (which may be either petroleum derived or biobased, as described herein) to form a reaction mixture; and reacting the reaction mixture to afford the bioderived polyester polymer. According to the various embodiments, the hydrogenolysis product may be produced by the hydrogenolysis of a bioderived polyol feedstock selected from glucose, sorbitol, glycerol, sorbitan, isosorbide, hydroxymethyl furfural, a polyglycerol, plant fiber hydrolyzates, fermentation products from plant fiber hydrolyzates, and mixtures of any thereof. The bioderived polyester polymer according to the various embodiments of the present method may be from 50% to 100% biobased (i.e., the polymer has a carbon isotope ratio characteristic of a material in which from 50% to 100% of the carbons are derived from biological sources). According to certain embodiments of the methods, wherein the saturated or unsaturated dicarboxylic acid monomer reagent is a bioderived saturated or unsaturated dicarboxylic acid monomer reagent, the resulting bioderived polyester polymer may be 100% biobased. The hydrogeriolysis product mixture may be used as a replacement for petroleum derived propylene glycol and/or petroleum derived ethylene glycol as a diol monomer reagent in the synthesis of polyester polymers.

In certain embodiments, the hydrogenolysis product may be mixed with a second biobased diol monomer reagent, as discussed herein, prior to mixing with the dicarboxylic acid monomer unit. In other embodiments, the method of making a bioderived polyester polymer may further comprise adding a modifier to the reaction mixture. Although any suitable modifier may be employed, according to certain embodiments, the modifier may be selected from furfural derivatives, such as, for example, a 2,5-dihydroxymethylfurfural derivative or 5-hydroxymethylfurfural derivative, or a diether derivative of 2,5-dihyroxylmethylfurfural or 5-hydroxymethylfurfural and either propylene glycol or ethylene glycol; difurfuryl ether; and mixtures of any thereof. The modifier according to any of these methods may be either biobased or derived from petroleum products. In certain methods comprising adding a modifier, the modifier may act by cross-linking adjacent polymer strands within the resultant polymer.

According to other embodiments, the method of making a bioderived polyester polymer wherein an unsaturated carboxylic acid monomer reagent is used to synthesize an unsaturated polyester polymer, may further comprise derivatizing or modifying the unsaturated polyester polymer using one or more chemical reactions. According to certain embodiments, the unsaturated polyester polymer may be derivatized as described herein. FIG. 1 illustrates one non-limiting approach to synthesizing and derivatizing one embodiment of an unsaturated polyester polymer.

In other embodiments, the present disclosure also contemplates methods for making a bioderived ester composition. The method may comprise reacting the hydrogenolysis product mixture, as described herein, with a fatty acid ester, such as a methyl ester, a carboxylic acid or a glyceride, such as a monoglyceride, a diglyceride, or a triglyceride. The reaction may result in a bioderived ester having a 100% biobased carbon isotope ratio. In certain embodiments, the hydrogenolysis product mixture may replace petroleum derived propylene glycol and/or ethylene glycol in the synthesis of esters of polyols.

For example, according to various embodiments of the method, the bioderived hydrogenolysis product mixture may be reacted, in place of petroleum derived propylene glycol, to form a biobased PGME, for example, by reacting the hydrogenolysis product mixture with a fatty acid ester or triglyceride by a transesterification reaction or with a fatty acid or other carboxylic acid by an esterification reaction.

According to other embodiments, the method may comprise reacting the hydrogenolysis product with lactic acid or a lactic acid derivative, such as a lactate ester, to form a mixed polyol lactate ester composition, as described herein. One non-limiting example of a synthesis of a mixed polyol lactate ester composition is set forth in FIG. 2.

In still another embodiment, the method may comprise reacting the hydrogenolysis product mixture with citric acid or a citric acid derivative to form a mixed polyol citrate ester composition, as described herein. One non-limiting example of a synthesis of a mixed polyol citrate ester composition is set forth in FIG. 3.

Various embodiments of the present disclosure will be better understood when read in conjunction with the following non-limiting Examples. The procedures set forth in the Examples below are not intended to be limiting herein, as those skilled in the art will appreciate that various modifications to the procedures set forth in the Examples, as well as to other procedures not described in the Examples, may be useful in practicing the invention as described herein and set forth in the appended claims.

### EXAMPLES

### Example 1

### Hydrogenolysis Process

Hydrogenolysis may include a fixed bed catalytic process that uses hydrogen at a pressure ranging from 1000 psi to 2000 psi, typically conducted at temperatures ranging from 180° C. to 250° C., and typically under alkaline conditions.

One embodiment is taught in U.S. Pat. No. 6,841,085, the disclosure of which is incorporated in its entirety by reference herein. A nickel-rhenium-on-carbon catalyst was loaded into a 300 mL semi-batch Parr reactor and purged with nitrogen. The catalyst was activated by adding hydrogen at 500 psi and heating to 280° C. for 16 h with stirring. The reactor was cooled, the hydrogen was removed, and 105.5 g or an aqueous solution of 25% sorbitol and 0.94% KOH was added. The reactor was pressurized with hydrogen to 600 psi and heated. When the temperature reached 220° C., the pressure was raised to 1200 psi. The reaction was allowed to proceed for 4 h. Depending on the catalyst compositions, sorbitol conversions ranged from 48.8% to 62.8%. In most cases, the major products were glycerol, propylene glycol, and ethylene glycol. Other feedstocks, including xylitol, arabinitol, lactic acid, and glycerol were also submitted to these reaction conditions.

Another embodiment is taught by U.S. Pat. No. 4,401,823, the disclosure of which is incorporated in its entirety by reference herein. Under these conditions, alditols (such as a 15-40%, by weight, solution of sorbitol in water) were catalytically hydrocracked in a fixed bed catalytic reaction process using an active nickel catalyst to produce at least about 30 wt. % conversion to glycerol and glycol products. The feedstream pH was controlled to between 4 and 14 by adding a basic promoter material such as calcium hydroxide to prevent damage to the catalyst. Reaction zone conditions were 400° F. to 500° F., 1200 psig to 2000 psig hydrogen partial pressure, and a liquid hourly space velocity of 1.5 to 3.0. To maintain the desired catalyst activity and product yields, the catalyst was regenerated to provide a catalyst age within the range of 20 h to 200 h. The reaction products were separated by distillations at successively lower pressures and unconverted alditol was recycled to the reaction zone for farther hydrogenolysis to produce 80 wt. % to 95 wt. % glycerol product. Sorbitol conversion was maintained within the range of 30 wt. % to 70 wt. % by catalyst regeneration following 20 h to 200 h use. Regeneration comprises washing the catalyst to remove deposits and beating with hydrogen at 500° F. to 600° F. Countercurrent flow of feed and hydrogen in the reaction zone can be used if desired, particularly for achieving higher conversions of alditol feed to glycerol products.

Another embodiment is taught by U.S. Pat. No. 6,982,328, the disclosure of which is incorporated in its entirety by reference herein. Under these conditions, catalytic hydrogenolysis utilizes a catalyst such as, for example a catalyst comprising a support and one or more metal catalysts including ruthenium, nickel, rhenium, and/or cobalt. The support can comprises for example, one or more of carbon, titania, and zirconia. Silicon dioxide or alumina are also suitable supports. Catalytic hydrogenolysis can further comprise utilization of an added base. For example, for neutral polyol feedstocks having a pH from about 5 to 8, such as sorbitol or glycerol, the appropriate pH for catalytic hydrogenolysis can be achieved by, for example, an addition of sodium hydroxide to a final concentration of from about 0% to about 10% by weight, or from about 0.5% to about 2% by weight, relative to the weight of the final solution.

### Example 2

### Plant Fiber Hydrolyzate for Polyol Feedstock

A plant fiber hydrolyzate from corn fiber was synthesized. The resulting hydrolyzate comprises polyol and polysaccharide residues suitable for a polyol feedstock for the hydrogenolysis process.

Corn fiber was obtained from Archer Daniels Midland Company (Decatur, Ill.) and subjected to hydrolysis. Thermochemical hydrolysis of corn fiber (65% moisture) obtained from a corn wet mill was carried out by treating 5 kg of corn fiber with steam in a rotating reactor at 145° C. for 30 minutes, Reaction mixtures were separated with a Rietz horizontal screw press (Minneapolis, Minn.) into a solid fraction comprising hydrolyzed corn fiber and a liquid fraction comprising corn fiber hydrolyzate. The solid fraction was then washed with 15 kg of water and the separated with the Rietz horizontal screw press. The wash liquid was pooled with the liquid corn fiber hydrolyzate. Nine batches of 5 kg of corn fiber were treated in this manner and the liquid fractions were pooled to obtain 193 kg of corn fiber hydrolyzate solution. About half of the corn fiber was rendered water soluble by this treatment. Corn fiber hydrolyzate solution (193 kg) was subjected to acid hydrolysis by addition of 1 wt-% sulfuric acid and heating to 121° C. for 30 minutes to yield an acid hydrolyzed corn fiber hydrolyzate. This solution was concentrated to yield approximately 44.4 kg of 31% (wt/wt) acid hydrolyzed corn fiber hydrolyzate concentrate containing 132 grains/liter of organic carbon. Part of the organic carbon (32.5 gram/L) was protein, and a trace (4.4 g/L) of acetic acid was noted. Concentrations of the residues of polysaccharides and polyols in the acid hydrolyzed corn fiber hydrolyzate concentrate are given in Table 2.

TABLE 2
Residues of polysaccharides and polyols obtained by hydrolysis
of corn fiber

| | Concentration | | | Hydroxyls | Moles |
|---|---|---|---|---|---|
| Polyol | (g/L) | MW | moles | per mol | Hydroxyl/Liter |
| Xylose | 53.60 | 144 | 0.37 | 5 | 1.85 |
| Arabinose | 48.50 | 144 | 0.34 | 5 | 1.70 |
| Fructose | 0.90 | 180 | 0.005 | 6 | 0.03 |
| Mannose | 1.40 | 180 | 0.008 | 6 | 0.048 |
| Galactose | 8.60 | 180 | 0.048 | 6 | 0.288 |
| Glucose | 57.00 | 180 | 0.317 | 6 | 1.90 |
| Sucrose | 0.08 | 344 | 0.0002 | 8 | 0.0016 |
| Maltose | 2.01 | 344 | 0.0058 | 8 | 0.046 |
| Total | 175.69 | | | | 5.8636 |
| Polyols | | | | | |

The resulting corn fiber hydrolyzate may be used as a polyol feedstock in a hydrogenolysis reaction according to the processes substantially as set forth in Example 1, for example the processes disclosed in U.S. Pat. No. 6,982,328.

### Example 3

### Fermentation Product of Plant Fiber Hydrolyzate as Polyol Feedstock

A fermentation product of a plant fiber hydrolyzate of corn fiber was synthesized. The resulting fermentation product comprises polyol and polysaccharide residues suitable for a polyol feedstock for the hydrogenolysis process.

Acid hydrolyzed corn fiber hydrolyzate concentrate from Example 2 was fermented by two Saccharomyces cerevisiae strains (ADM y500, available from Archer Daniels Midland Company, Decatur, Ill., and r424a, an experimental strain obtained from the Laboratory of Renewable Resources Engineering at Purdue University, West Lafayette, Ind.) in two separate continuous fermentations in a set of four New Brunswick BioFlo III fermentors (Edison, N.J.) with a working volume of 2100 mL. The initial fermentation volume in each fermentor was 1500 mL and yeast inoculum was 10%. In one, the fermentation medium was composed of 40% “blender mix” (liquefied starch, backset, corn steep liquor) (from Archer Daniels Midland Company, Decatur, Ill.) and 60% acid hydrolyzed corn fiber. All four fermentors were inoculated with r424a (LNHst).

In the second set of four fermentors, the fermentation medium was composed of 80% blender mix and 20% acid hydrolyzed corn fiber hydrolyzate. In the second set, two fermentors were inoculated with ADM y500 and two were inoculated with r424a (LNHst). Amyloglucosidase (EC 3.2.1.3, available from Archer Daniels Midland Company, Decatur, Ill.), 1 ml, per liter of fermentation media, was added to the fermentors at the start of the fermentation to hydrolyze any maltooligosaccharides remaining in the corn fiber hydrolyzate. The fermentations were run without air addition at 31° C., pH 4.5 (controlled by ammonium hydroxide addition), and agitation, which was carried out with a single impeller at 150 rpm. The only feeds to the fermentors were blender mix, corn fiber liydrolyzate, and ammonium hydroxide. Samples were taken periodically and the concentrations of polyols were determined by HPLC. The spent fermentation media from both fermentation runs (8 fermentors total) were combined and centrifuged to remove the cell mass and solids. The liquid portion was evaporated in a forced circulation, long-tube vertical evaporator to remove ethanol and some water, to provide a solution containing residues of polysaccharides and polyols (Table 3).

The resulting fermetation product of the corn fiber hydrolyzate may be used as a polyol feedstock in a hydrogenolysis reaction according to the processes substantially as set forth in Example 1.

TABLE 3
Residues of polysaccharides and polyols from fermented acid
hydrolyzed corn fiber hydrolyzate

| | Concentration | | | Hydroxyls | Moles |
|---|---|---|---|---|---|
| Polyol | (grams/Liter) | MW | moles | per mol | Hydroxyl/Liter |
| Arabinose | 32.6 | 144 | 0.226 | 5 | 1.13 |
| Xylose | 28.6 | 144 | 0.199 | 5 | 0.995 |
| Sucrose | 0.3 | 344 | 0.0009 | 8 | 0.0072 |
| Maltose | 0.2 | 344 | 0.0006 | 8 | 0.0048 |
| Isomaltose | 1.9 | 344 | 0.0055 | 8 | 0.044 |
| Fructose | 0.8 | 180 | 0.004 | 6 | 0.024 |
| Mannose | 0.5 | 180 | 0.003 | 6 | 0.018 |
| Galactose | 6.9 | 180 | 0.038 | 6 | 0.228 |
| Glucose | 8.9 | 180 | 0.049 | 6 | 0.294 |
| Total | 80.7 | | | | 2.745 |

### Example 4

### Latex Paint Formulation

In this Example, a latex paint formulation wherein petroleum derived propylene glycol has been replaced with product mixture from the hydrogenolysis of glycerol and/or esters of the product mixture from the hydrogenolysis of glycerol.

A composition enriched in compounds containing 2 hydroxyl groups was obtained by hydrogenolysis of glycerol produced by passing a 40% solution of crude glycerol through a reactor substantially as set forth in Example 1. The crude glycerol was obtained as by-product of palm biodiesel synthesis. The hydrogenolysis product was dewatered by distillation. A composite product was prepared by combining four dewatered glycerol hydrogenolysis product samples to yield a mixture of polyols containing 75.5% propylene glycol, 4.5% ethylene glycol, 18% lactic acid, 12.2% glycerol, and 0.5% water. This composition was subjected to short path distillation to reduce the water content to 0.15% and the undistilled residue enriched in compounds containing two hydroxyl groups (Mixture 1) had the following composition: 75.8% propylene glycol, 4.7% ethylene glycol, 1.8% lactic acid, 1.3% 2,3-butanediol, and 13.8% glycerol.

Fatty acid esters of Mixture 1 were synthesized as follows. Mixture 1 (pH ˜8, 150 g) was mixed with corn oil (130 g) (commercially available from Archer-Daniels-Midland Company, Decatur, Ill., and other sources) and 0.98 g sodium methoxide in a Parr reactor. The reactor was purged with nitrogen and heated to ftom 210° C. to 220° for 3 hrs. After cooling, the solution was neutralized with 3.5 g citric acid. Thin layer chromatography (silica gel 60 plates developed with 1:1 ethyl ether:hexane and stained with sulfuric acid) showed spots consistent with the desired product and very little remaining starting material. The reaction product containing the fatty acid esters of Mixture 1 was mixed with ether and allowed to settle overnight, after which the ether layer had become transparent. The ether was removed from the upper layer by vacuum rotary evaporation to yield a translucent yellow liquid having the composition shown in Table 4 (“Mixture 1-FA esters”).

TABLE 4
Composition of Fatty Acid Esters from Glycerol Hydrogenolysis Product

| Component | Percent (% wt) |
|---|---|
| PGME | 55.46% |
| 1,2-Propanediol | 19.31% |
| Di-PGME | 10.37% |
| Propylene glycol diester (PGDE) | 5.93% |
| Fatty acids | 3.70% |
| Glycerol | 1.77% |
| Di-PGDE | 1.40% |
| Water | 0.76% |
| Ethylene glycol | 0.63% |
| 2,3-Butanediol | 0.37% |
| Dipropylene glycol | 0.30% |

Mixture 1 was used to replace petroleum derived propylene glycol component in the Grind portion of a latex paint formula and the composition comprising the fatty acids esters of Mixture I (Mixture 1-FA esters) was used to replace Archer RC® (petroleum derived PGME, commercially available from Archer-Daniels-Midland Company, Decatur, Ill.) in the Let down portion of the latex paint formulation. Four latex paint formulations (low sheen interior/exterior white paint with <50 g/L, VOC) comprising Mixture I as a replacement for petroleum derived propylene glycol and/or Mixture 1-FA esters as a replacement for Archer RC® were prepared and compared to a control comprising petroleum derived propylene glycol and Archer RC®. In Formula A petroleum derived propylene glycol in the Grind portion was replaced with biobased Mixture 1. Formula B contained propylene glycol in the Grind portion but replaced the Archer RC® in the Let down portion with Mixture 1-FA esters. Formula C contained propylene glycol in the Grind portion by replaced the Archer RC® in the Let down portion with twice the amount of the Mixture 1-FA esters (as compared to Formula B). Formula D replaced the petroleum derived propylene glycol in the Grind portion with biobased Mixture 1 and the Archer RC® in the Let down portion with twice the amount of the Mixture 1-FA esters. Table 5 presents the four latex paint formulations containing biobased products and a control formulation that contained petroleum based products.

The latex paint formulations were prepared as follows. For the Grind portion, the Grind ingredients were added one at a time while mixing under low speed (200-300 rpm) with a high speed disperser (Stir Pak or Hockmeyer). When all grind ingredients were added, the speed was increased to 800-1200 rpm to completely disperse the pigment to a 5-6 NS fineness of grind. For the Let down portion, the Let down ingredients were added one at a time while mixing under medium speed (600-800 rpm) to complete the paint. After all ingredients had been added, mixing was continued for about 15 minutes.

TABLE 5
Latex Paint Formulations (Grind and Let down Portions)

| | | Control | Formula A | Formula B | Formula C | Formula D |
|---|---|---|---|---|---|---|
| | | Pounds | Pounds | Pounds | Pounds | Pounds |
| Raw Material | | (lbs) | (lbs) | (lbs) | (lbs) | (lbs) |
| Grind | Water | 70.00 | 70.00 | 70.00 | 70.00 | 70.00 |
| | Propylene glycol | 12.00 | — | 12.00 | 12.00 | — |
| | Mixture 1 | — | 12.00 | — | — | 12.00 |
| | Tamol 1124 | 5.00 | 5.00 | 5.00 | 5.00 | 5.00 |
| | Omyacarb UF | 165.00 | 165.00 | 165.00 | 165.00 | 165.00 |
| | Kathon LX 1.5% | 1.75 | 1.75 | 1.75 | 1.75 | 1.75 |
| Let | TiO₂ Slurry | 260.00 | 260.00 | 260.00 | 260.00 | 260.00 |
| Down | Water | 60.00 | 60.00 | 60.00 | 60.00 | 60.00 |
| | Rhoplex SG-30 | 440.00 | 440.00 | 440.00 | 440.00 | 440.00 |
| | Archer RC | 11.27 | 11.27 | — | — | — |
| | Mixture 1-FA esters | — | — | 11.27 | 22.54 | 22.54 |
| | Aerosol OT-75 | 1.50 | 1.50 | 1.50 | 1.50 | 1.50 |
| | BYK 1660 | 2.06 | 2.06 | 2.06 | 2.06 | 2.06 |
| | Ammonia (28%) | 1.50 | 1.50 | 1.50 | 1.50 | 1.50 |
| | Acrysol RM | 16.00 | 16.00 | 16.00 | 16.00 | 16.00 |
| | 2020NPR | | | | | |
| | Acrysol SCT-275 | 6.00 | 6.00 | 6.00 | 6.00 | 6.00 |
| | Water | 48.98 | 48.98 | 48.98 | 48.98 | 48.98 |
| Total | | 1101.06 | 1101.06 | 1101.06 | 1112.33 | 1112.33 |

Tamol 1124 is a sodium carboxylate dispersant (Rohm & Haas, Philadelphia, PA)
Omyacarb UF is a high purity, ultrafine, wet ground calcium carbonate (Omya Inc., Proctor, Vt.)
Kathon LX is a biocide latex paint preservative (Rohm & Haas, Philadelphia, PA)
Rhoplex SG-30 is an acrylic binder (Rohm & Haas, Philadelphia, PA)
Aerosol OT-75 is an anionic surfactant (Cytec Industries, West Paterson, NJ)
BYK 1660 is an emulsion of siloxylated polyethers (BYK-Chemie, Wallingford, CT)
Acrylsol RM 2020NPR is a modified ethylene oxide urethane rheology modifier (Rohm & Haas, Philadelphia, PA)
Acrysol SCT-275 is a rheology modifier (Rohm & Haas, Philadelphia, PA)

The latex paint formulations were tested for viscosity, pH, curing, gloss, opacity, open time, color, stability, freeze-thaw stability (using ASTM D2243), scrub cycles (using ASTM D2486) and block test (using ASTM D4946). The results are presented in Table 6.

TABLE 6
Properties of Latex Paint Formulations

| Paint Properties | | Control | Formula A | Formula B | Formula C | Formula D |
|---|---|---|---|---|---|---|
| Viscosity, ku/ICI | | 103.3/0.80 | 104.6/0.75 | 98.1/0.746 | 116.6/0.746 | 117.7/0.738 |
| pH | | 9.49 | 9.25 | 9.19 | 9.39 | 9.32 |
| Curing @ 40° F. | | passed | passed | failed | passed | passed |
| Gloss @ 60 deg | | 21.5 | 20.7 | — | 24.4 | 24 |
| Opacity | | 96.36 | 96.56 | — | 96.7 | 96.55 |
| Open Time¹ | | Standard | Equal to | — | Equal to | Equal to |
| | | | Standard | | Standard | Standard |
| Color - CIELab | | | | | | |
| Lightness L | | 96.49 | 96.21 | — | 96.38 | 96.36 |
| Yellowness b | | 1.67 | 1.65 | | 1.66 | 1.69 |
| Yellowing Index YE | | 2.42 | 2.40 | | 2.41 | 2.46 |
| Heat-aged Stability | | | | | | |
| 10 days @140° F. | | | | | | |
| Δ pH | | −0.19 | +0.10 | — | −0.29 | −0.15 |
| Δ Viscosity, ku | | +4.90 | +5.60 | | −7.20 | −7.20 |
| Δ Gloss @ 60 deg | | 0 | +1.80 | | +1.80 | +2.60 |
| CIELab, Δb yellowness | | 0.14 | 0.21 | | 0.10 | 0.11 |
| ΔYE, yellowing index | | 0.43 | 0.33 | | 0.16 | 0.17 |
| Freeze-thaw ASTM | | failed | failed | — | failed | failed |
| D2243 @ Cycle 1 | | | | | | |
| Scrub Cycles ASTM | | 1600 | 1600 | 1567 | 1881 | 1975 |
| D2486 | | | | | | |
| Block Test ASTM D4946 | | | | | | |
| 120° F. | 1 day cure | 3 | 2 | — | 1 | 2 |
| | 7 day cure | 8 | 8 | | 9 | 8 |

¹Resin vendor in-house test procedure

Latex paint in which Mixture 1 replaced petroleum derived propylene glycol in the Grind portion (Formula A) demonstrated open time, block test and scrub resistant equivalent to that of the control formulation made with petroleum derived propylene glycol. Formula A demonstrated a slight increase in yellow before and after the heat-aged stability test which may be attributed to the initial amber color of Mixture 1. The degree of failure in the freeze-thaw stability of Formula A was equal to that of the control formulation.

Latex paint with which Archer RC® in the Let down phase was replaced with an equal weight of Mixture 1-FA esters (Formula B) resulted in a latex paint that failed the low temperature curing test (at 40° F.). Consequently, no further evaluation was performed on this formulation.

Latex paint with which Archer RC® in the Let down phase was replaced with an twice the weight of Mixture 1-FA esters (Formula C) resulted in a latex paint that passed the low temperature curing test and demonstrated greater viscosity and gloss than the control formulation. Formula C also gave better scrub resistance than the control. Block resistance of Formula C after one day was less than the control formulation, but as the paint hardened during seven days of curing at elevated temperature, the paint film became harder than the control formulation. In addition, Formula C also displayed a decrease in pH and gloss after the 10-day heat-aged stability test at 140° F.

For latex paint Formula D where the petroleum derived propylene glycol in the Grind phase was replaced with biobased Mixture 1 and the Archer RC® in the Let down phase was replaced with Mixture 1-FA esters (at twice the weight content of the Archer RC®), the resulting latex paint formulation displayed higher viscosity, gloss, and scrub resistance than the control formulation. In addition, the block resistance of Formula D was comparable with that of the control formulation.

Properties of latex paint formulations in which petroleum derived propylene glycol was replaced with biobased Mixture 1 and/or petroleum derived Archer RC® was replaced with biobased Mixture 1-FA esters (at twice the amount of the Archer RC®) displayed properties that were equal to or substantial improvements of the properties of the control formulation. The biobased hydrogenolysis product mixtures may be used as replacement of petroleum based products in the formulation of latex paints.

### Example 5

### Polyester Polymerization Reaction

This Example sets forth a representative polyester polymerization reaction using a hydrogenolysis product mixture obtained by hydrogenolysis of glycerol or sorbitol according to certain embodiments disclosed herein.

A composition enriched in compounds containing two hydroxyl groups was obtained by hydrogenolysis of glycerol by passing a 40% solution of crude glycerol obtained as a by-product of a palm biodiesel synthesis through a reactor substantially as set forth in Example 1. The reactor product was dewatered by distillation. A composite was prepared by combining four dewatered glycerol hydrogenolysis product samples to yield a mixture of polyols having the composition: 75.5% propylene glycol, 4.5% ethylene glycol, 1.8% lactic acid, 12.2% glycerol, and 0.5% water. This composition was subjected to short path distillation to reduce the water content to 0.15% and the undi stilled residue enriched in compounds containing two hydroxyl groups (Mixture 1) had the following composition: 75.8% propylene glycol, 4.7% ethylene glycol, 1.8% lactic acid, 1.3% 2,3-butanediol, and 13.8% glycerol.

In one study, the composition enriched in compounds containing two hydroxyl groups is combined with an equimolar quantity of diisocyanate to make a predominantly linear polyurethane using the procedure set forth by Frisch (“Fundamental Chemistry and Catalysis of Polyirrethanes,” Frisch, K. C., in Polyurethane Technology, Paul Bruins, ed., Interscience Publishers, New York, 1969, the disclosure of which is incorporated in its entirety by reference herein).

In a second study, the hydrogenolysis product from the hydrogenolysis of sorbitol containing, by weight percent, 0.25% glucose; 0.25% xylose; 0.25% arabinose; 1.74% arabitol; 1.24% erythritol; 6.47% lactate; 10.45% glycerol; 1.00% 1,2,4-butanetriol; 42.54% ethylene glycol; 32.34% propylene glycol; 1.00% 2,3-butanediol; 0.50% 1,3-butanediol; and 2.00% 1,2-butanediol is combined with a diisocyanate at 100° C. to make a branched polymer.

The polymers resulting from study 1 and 2 will be suitable for use in fibers, hard and soft elastomers, coatings and adhesives, flexible and rigid foams, and thermoplastics and thermosetting plastics.

### Example 6

### Sythesis of a Polyol Ester

This Example sets forth a representative synthesis of a polyol ester mixture from vegetable oils and the hydrogenolysis product mixture obtained by hydrogenolysis of sorbitol.

A polyol sample (200 g) from the hydrogenolysis of sorbitol containing, by weight percent, 0.25% glucose; 0.25% xylose; 0.25% arabinose; 1.74% arabitol; 1.24% erythritol; 6.47% lactate; 10.45% glycerol; 1.00% 1,2,4-butanetriol; 42.54% ethylene glycol; 32.34% propylene glycol; 1.00% 2,3-butanediol; 0.5% 1,3-butanediol; and 2.00% 1,2-butanediol was combined with dried corn oil (200 g) and sodium inethoxide (1.0 g) in a 1000 mL round bottom flask. The mixture was heated with agitation at 120° C. for 4 hours. The product was cooled and neutralized with citric acid. Hexane was added and the organic layer was recovered. The hexane was removed from the product using a rotary evaporator under reduced pressure to give a residue of polyol esters of corn oil fatty acids, If desired, the product can be stripped using a wiped film evaporator/miiolecular still at 90° C., 0.6 millibars, 270 rpm and a flow rate of 4 mL/min. The resulting polyol ester composition is suitable for use as a 100% biobased replacement for a petroleum derived propylene glycol monoester.

### Example 7

### Synthesis of PGME Enriched Polyol Esters of Soy Oil

This Example sets forth a representative synthesis of a propylene glycol monoester from a vegetable oil and the hydrogenolysis product mixture from the hydrogenolysis of sorbitol.

Sorbitol was subjected to hydrogenolysis substantially as set forth in Example 1. The hydrogenolysis product was then subjected to distillation to remove the water. The compositions of the hydrogenolysis product before and after stripping are set forth in Table 7.

TABLE 7
Composition of Hydrogenolysis Product

| | Hydrogenolysis product | Hydrogenolysis product |
|---|---|---|
| Compound | before stripping (wt %) | after stripping (wt %) |
| Sorbitol | 6.2% | 10.0% |
| Xylitol | 2.2% | 3.5% |
| Erthyritol | 0.8% | 1.3% |
| Lactate | 1.0% | 1.6% |
| Glycerol | 10.9% | 17.6% |
| 1,2,4-Butanetriol | 0.5% | 0.8% |
| Ethylene glycol | 11.4% | 18.4% |
| Propylene glycol | 22.3% | 36.0% |
| 2,3-Butanediol | 1.4% | 2.3% |
| 1,3-Butanediol | 1.0% | 1.6% |
| 1,2-Butanediol | 2.5% | 4.0% |
| Ethanol | 0.4% | 0.6% |
| Isopropanol | 0.2% | 0.3% |
| Water | 38.0% | 0% |
| Unknown | 1.2% | 1.9% |

A 1 liter autoclave reactor was charged with RBD soybean oil (refined, bleached, and deodorized soybean oil, 160 g), the hydrogenolysis product mixture from sorbitol (165 g), potassium acetate (0.08 g), and lithium hydroxide (0.02 g). The reactor headspace was purged with nitrogen. The reactor was pressurized with nitrogen at 350 psi and agitation at 800 rpm was began. The reaction mixture was heated to 240° C. over 1 hour at which time the pressure has increased to 550 psi. The reaction was held at 240° C. for 1.5 hours and then rapidly cooled to room temperature. The contents of the reactor were placed in a separatory funnel and neutralized with 0.5 g of conc. H₃PO₄, The mixture was extracted with hexanes and the organic layer was washed once with four times its volume of deionized water. The organic layer was dried over anhydrous magnesium sulfate and filtered. The filtrate was concentrated with a rotary evaporator under reduced pressure to give a product having a Lovibond color of 2.9R., 14.0Y. The product composition was 60-81% propylene glycol monoester and 5% propylene glycol diester with an acid value of 21.6. This material may be used as a 100% biobased polyol ester replacement for petroleum derived PGMEs, for example as a coalescent in a latex paint formulation.

### Example 8

### Candle Wax Esters

This Example sets forth a representative synthesis of a waxy propylene glycol monoester from a hydrogenated vegetable oil and the hydrogenolysis product mixture.

A 1 L three neck round bottom flask was fitted with a heating mantle, a magnetic stirrer, a reflux condenser, and nitrogen sparge. Sorbitol was subjected to hydrogenolysis to obtain a hydrogenolysis product containing polyols (before stripping) and a composition as recited in Table 7, then heated under vacuum in a rotary evaporator to remove water and lower molecular weight alkyl monohydroxyl alcohols to obtain a stripped mixed polyol sorbitol hydrogenolysis product mixture (Table 7). The reaction vessel was charged with melted soy titer (fully hydrogenated soybean oil, 150 g) and the stripped mixed polyol mixture from the hydrogenolysis of sorbitol (30 g). The mixture was heated to 150° C. with agitation and NaOH (0.18 g) was added to catalyze alcoholysis of the melted soy titer by the polyol mixture. The mixture was heated from 150° C. to 220° C. with nitrogen sparging and good agitation over 1 hour. The product mixture enriched in fatty acid esters of polyols was then quickly cooled and neutralized with cone. H₃PO₄ (0.55 g). The cooled, neutralized product mixture separated into an upper phase containing the fatty acid esters of polyols and remaining titer esters and an aqueous bottom phase and the top phase solidified at room temperature. The solid top phase was collected and used in as a wax in a biobased candle wax formulation.

### Example 9

### Synthesis of Levulinate Esters

In this Example levulinate derivatives of residues of polysaccharides and polyols derived from hydrogenolysis of sorbitol were synthesized.

A glycerol hydrogenolysis product was produced by passing a 40% solution of crude glycerol obtained as a by-product of a palm biodiesel synthesis through a 1000 milliliter trickle bed reactor containing a nickel-rhenium-on-carbon catalyst. The pH of the glycerol solution was made alkali by addition of sodium hydroxide (1.3%). Hydrogen gas and the liquid phase were preheated to 195° C. to 230° C. and fed co-currently into the reactor at 1 linear hourly space velocity (LHSV), which was operated at 1200 psi. The liquid phase feed rate was 30 mL/minute and the reactor temperature was 210° C. The reactor product comprising the hydrogenolysis product of glycerol was collected in a high-pressure pot. The hydrogenolysis product was dewatered by distillation. A composite was prepared by combining four hydrogenolysis product samples to yield a mixture of polyols comprising 75.5% propylene glycol, 4.5% ethylene glycol, 1.8% lactic acid, 12.2% glycerol, and 0.5% water.

This mixture of polyols (25 g) derived from the hydrogenolysis product of glycerol was combined with levalinic acid (83.5 g) and dry Amberlyst 36 resin (10 g, commercially available from Rohm & Haas Co., Woodridge, Ill.). Amberlyst 36 is a macroreticular, strongly acidic, polymeric resin catalyst. This mixture was heated with stirring to 115° C. under vacuum (30 mmHg) for 1 hour. A total of 10.5 mL, of water was collected in the vacuum receiver. The solution was filtered to remove the resin. The filtered product (76.7 g) comprised 1.8% propylene glycol, <0.1% ethylene glycol, and <0.1% glycerol, and 92.6% polyol levulinate esters.

### Example 10

### Mixed Polyol Lactate Esters

This Example sets forth a representative synthesis of a mixture of polyol lactate esters from ethyl lactate and the hydrogenolysis product mixture. The hydrogenolysis product mixture of polyols from the hydrogenolysis of glycerol as described in example 9 (25 g) is combined with lactic acid (65 g) and sodium methoxide (1 g). The esterification of a mixture of polyols with lactic acid is performed by heating lactic acid with the mixed polyols substantially as in example 9. After filtering to remove the resin, a product comprising lactate esters of the mixture of polyols from the hydrogenolysis of glycerol is obtained.

### Example 11

### Mixed Polyol Citrate Esters

This example sets forth a representative synthesis of a mixture of polyol citrate esters from citric acid and the hydrogenolysis product mixture. The mixture of polyols from hydrogenolysis of glycerol and used in example 9 (33 g) is combined with citric acid (50 g) and dry Amberlyst 36 resin (10 g, Rohm and Haas Co., Woodridge, Ill.). The esterification of a mixture of polyols with citric acid is performed by heating citric acid with the mixed polyols substantially as in example 9. The reaction mixture is filtered to remove the resin and a product comprising citrate esters of the mixture of polyols from the hydrogenolysis of glycerol is obtained.

Although the foregoing description has presented a number of embodiments of the invention, those of ordinary skill in the relevant art will appreciate that various changes in the components, details, materials, and process parameters of the examples that have been herein described and illustrated in order to explain the nature of the invention may be made by those skilled in the art, and all such modifications will remain within the principle and scope of the invention as expressed herein in the appended claims. It will also be appreciated by those skilled in the art that changes could be made to the embodiments described above without departing from the broad inventive concept thereof. It is understood, therefore, that this invention is not limited to the particular embodiments disclosed, but it is intended to cover modifications that are within the principle and scope of the invention, as defined by the claims.

1. A composition comprising:
a hydrogenolysis product of a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof,
wherein the hydrogenolysis product comprises a mixture of propylene glycol, ethylene glycol, and one or more of methanol, 2-propanol, glycerol, lactic acid, glyceric acid, butanediols, sodium lactate, and sodium glycerate,
wherein the composition is 100% biobased as determined by ASTM International Radioisotope Standard Method D 6866.

2. The composition of claim 1, wherein the hydrogenolysis product comprises 0.1% to 99.9% by weight of propylene glycol, 0.1% to 99.9% by weight of ethylene glycol, 0% to 99.9% by weight of methanol, 0% to 99.9% by weight of 2-propanol, 0% to 99.9% by weight of glycerol, 0% to 99.9% by weight of lactic acid, 0% to 99.9% by weight of glyceric acid, 0% to 99.9% by weight of butanediols, 0% to 99.9% by weight of sodium lactate, and 0% to 99.9% by weight of sodium glycerate.

3. The composition of claim 2, wherein the hydrogenolysis product is purified by a purification method selected from the group consisting of chromatography, extraction, distillation, electrodialysis, and combinations of any thereof.

4 The composition of claim 2, wherein the hydrogenolysis product is purified by ion exclusion chromatography.

5. The composition of claim 1, wherein the composition is a diol reagent in a polyester polymerization reaction.

6. The composition of claim 5, wherein the polyester polymerization reaction is an unsaturated polyester polymerization reaction.

7. The composition of claim 5, wherein the diol reagent reacts with a dicarboxylic acid reagent selected from the group consisting of a petroleum derived saturated dicarboxylic acid, a petroleum derived unsaturated dicarboxylic acid, a bioderived saturated dicarboxylic acid, a bioderived unsaturated dicarboxylic acid, and mixtures of any thereof.

8. The composition of claim 5, wherein the diol reagent reacts with a bioderived unsaturated dicarboxylic acid selected from the group consisting of fumaric acid, 2,5-furandicarboxylic acid, a C₁₈- to C₂₄-unsaturated dicarboxylic acid, a dimerized unsaturated fatty acid, an unsaturated polycarboxylic acid, and mixtures of any thereof.

9 The composition of claim 5, wherein the diol reagent reacts with a bioderived saturated dicarboxylic acid selected from the group consisting of succinic acid, tetrahydrofuran-2,5-dicarboxylic acid, a C₁₈- to C₂₄-saturated dicarboxylic acid, a dicarboxylic acid derived from the ozonolysis of a vegetable oil, a dimerized saturated fatty acid, a saturated polycarboxylic acid, and mixtures of any thereof.

10. The composition of claim 5, wherein the diol reagent is further mixed with a second biobased diol reagent selected from the group consisting of tetrahydro-2,5-furandimethanol, 2,5-furandimethanol, 1 ,3-propanediol, 1,18-octadecanediol, 1,9-octadecanediol, 1,10-octadecanediol, a fatty alcohol dimer, isosorbide, isomannide, and mixtures of any thereof.

11. The composition of claim 5, wherein the polyester polymerization reaction further comprises a modifier selected from the group consisting of a 5-hydroxymethylfurfural derivative, a 2,5-dihydroxymethylfurfural derivative, a furfural derivative, difurfuryl ether, and mixtures of any thereof.

12-13. (canceled)

14. The composition of claim 13, wherein the hydrogenolysis product is reacted with one of a fatty acid methyl ester and a triglyceride to form the propylene glycol monoester or diester.

15. The composition of claim 14, wherein the hydrogenolysis product is reacted with a triglyceride selected from the group consisting of corn oil, soybean oil, canola oil, vegetable oil, safflower oil, sunflower oil, nasturtium seed oil, mustard seed oil, olive oil, sesame oil, peanut oil, cottonseed oil, rice bran oil, babassu nut oil, castor oil palm oil, palm kernel oil, rapeseed oil, low erucic acid rapeseed oil, lupin oil, jatropha oil, coconut oil, flaxseed oil, evening primrose oil, jojoba oil, tallow, beef tallow, butter, chicken fat, lard, dairy butterfat, shea butter, biodiesel, used frying oil, oil miscella, used cooking oil, yellow trap grease, hydrogenated oils, derivatives of these oils, fractions of these oils, conjugated derivatives of these oils and mixtures of any thereof.

16. A candle wax formulation comprising the composition of claim 14.

17-18. (canceled)

19. A de-icing product formulation comprising the composition of claim 1.

20. A latex paint formulation comprising the composition of claim 1.

21. A method of making a bioderived composition for use as a replacement for petroleum derived propylene glycol or ethylene glycol, the method comprising:
reacting a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof, via a hydrogenolysis process to give a hydrogenolysis product comprising a mixture of propylene glycol, ethylene glycol, and one or more of methanol, 2-propanol, glycerol, lactic acid, glyceric acid, butanediols, sodium lactate, and sodium glycerate, wherein the hydrogenolysis product is 100% biobased as determined by ASTM International Radioisotope Standard Method D 6866; and
adding the hydrogenolysis product to a formulation as a replacement for petroleum derived propylene glycol or ethylene glycol.

22. (canceled)

23. The method of claim 21, wherein the formulation is a latex paint formulation, further comprising: purifying the hydrogenolysis product by a purification process selected from the group consisting of chromatography, electrodialysis, extraction, and distillation, prior to adding to the latex paint formulation.

24. A method for making a bioderived polyester polymer, the method comprising:
mixing a hydrogenolysis product with one of a bioderived saturated dicarboxylic acid monomer reagent and an unsaturated dicarboxylic acid monomer reagent to form a reaction mixture; and
reacting the reaction mixture to afford the bioderived polyester polymer,
wherein the hydrogenolysis product is produced by hydrogenolysis of a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof, and comprises a mixture of propylene glycol, ethylene glycol, and one or more of methanol, 2-propanol, glycerol, lactic acid, glyceric acid, butanediols, sodium lactate and sodium glycerate, and
wherein the bioderived polyester polymer is from 50% to 100% biobased as determined by ASTM International Radioisotope Standard Method D 6866.

25. The method of claim 24, wherein the unsaturated dicarboxylic acid monomer reagent is a bioderived unsaturated dicarboxylic acid monomer reagent, and
wherein the bioderived polyester polymer is 100% biobased as determined by ASTM International Radioisotope Standard Method D 6866.

26. The method of claim 24, further comprising adding a bioderived modifier to the reaction mixture, wherein the modifier is selected from the group consisting of a 2,5-hydroxymethylfurfural derivative, a furfural derivative, and mixtures of any thereof.

27. A method for making a bioderived ester, the method comprising:
reacting a hydrogenolysis product with one of a fatty acid methyl ester, a carboxylic acid, and a triglyceride,
wherein the hydrogenolysis product is produced by hydrogenolysis of a bioderived polyol feedstock selected from the group consisting of glucose, sorbitol, glycerol, sorbitan, isosorbide, hydroxymethyl furfural, a polyglycerol, a plant fiber hydrolyzate, a fermentation product from a plant fiber hydrolyzate, and mixtures of any thereof, and comprises a mixture of propylene glycol, ethylene glycol, and one or more of methanol, 2-propanol, glycerol, lactic acid, glyceric acid, butanediols, sodium lactate, and sodium glycerate, and
wherein the bioderived ester is 100% biobased as determined by ASTM International Radioisotope Standard Method D 6866.

28. The method of claim 27, wherein the hydrogenolysis product is reacted to form a propylene glycol monoester.

29. The method of claim 27, wherein the hydrogenolysis product is reacted with a lactic acid derivative to form a mixed polyol lactate ester composition.

30. The method of claim 27, wherein the hydrogenolysis product is reacted with a citric acid derivative to form a mixed polyol citrate ester composition.
